(19) United States
(12) Patent Application Publication
(10) Pub. No.: US 2022/0096602 A1
(43) Pub. Date: Mar. 31, 2022

(54) LIF THERAPY FOR INDUCING INTESTINAL EPITHELIAL CELL REGENERATION

(71) Applicant: Rutgers, the State University of New Jersey, New Brunswick, NJ (US)

(72) Inventors: Wenwei HU, Belle Mead, NJ (US); Zhaohui FENG, Belle Mead, NJ (US); Huaying WANG, Ningbo, Zhejiang (CN); Jianming WANG, Edison, NJ (US)

(21) Appl. No.: 17/432,177

(22) PCT Filed: Feb. 27, 2020
(86) PCT No.: PCT/US2020/020141

§ 371 Date: Aug. 19, 2021

Related U.S. Application Data
(60) Provisional application No. 62/811,396, filed on Feb. 27, 2019.

(51) Int. Cl. A61K 38/20; A61P 1/00; A61P 37/06; C12N 5/071; A61K 9/00

(52) U.S. Cl. CPC A61K 38/2093; A61P 1/00; C12N 2501/235; C12N 5/0679; A61K 9/0053; A61P 37/06

(57) ABSTRACT

Disclosed herein are new therapeutic methods and compositions for maintaining and regenerating intestinal epithelial cells in a mammal. In a specific embodiment, the methods involve administering an amount LIF that induces intestinal epithelial cell regeneration. The methods and compositions are useful for preventing or treating gastrointestinal radiation injury or GVHD.

### CROSS-REFERENCE TO RELATED APPLICATION

This application claims the benefit of U.S. Provisional Patent Application No. 62/811,396, filed Feb. 27, 2019, which is incorporated by reference herein in its entirety.

### STATEMENT OF GOVERNMENTAL INTEREST

This invention was made with Government support under Contract No. CA160558 and CA227912 awarded by the National Institute of Health. The Government has certain rights in the invention.

### BACKGROUND

### 1. Field of the Invention

This invention relates generally to the fields of medicine and veterinary medicine, and particularly to methods for treating gastrointestinal radiation injury, accidental radiation injury and GvHD by administering therapeutically effective amounts of LIF or variant thereof or a biologically active fragment of LIF or variant thereof to treat the subject.

### 2. Background of the Invention

Gastrointestinal radiation injury (GRI) is caused by exposure to deleterious levels of ionizing radiation for any reason, including administration as a treatment for different types of cancers, bone marrow transplantation or accidental exposure. Damage from the therapeutic use of ionizing radiation is a major problem and there is a need for more effective treatments.

Graft-versus-host disease (GvHD) is a medical complication that often occurs following the receipt of nonautologous transplanted tissue from a genetically different person. GvHD is commonly associated with stem cell transplants such as those that occur with bone marrow transplants. Heavy amounts of IR are administered to eradicate cancer cells in the bone marrow before a bone marrow transplant which can severely compromise the patient and result in graft. vs. host disease (GVHD). GvHD also occurs after other forms of tissue transplants such as solid organ transplants. White blood T-cells (herein T-cells) of the donor's immune system that remain within the donated tissue (the graft) recognize the recipient (the host) as foreign (non-self). The white blood T-cells present within the transplanted tissue then attack the recipient's own cells, which leads to GvHD. This problem is widespread, and there is a need for more effective treatments.

GRI and GvHD are particularly problematic to treat or prevent when the subjects (cancer patients, BMT recipients) are not healthy in the first place. Gastrointestinal diseases further includes a broad range of disorders related to the digestive tract that usually involve damage to the intestinal epithelium. For example, common gastrointestinal diseases include inflammation and ulcers of any part of the digestive tract such as the esophagus, stomach, or duodenum. A significant fraction of the world population experiences one or more gastrointestinal diseases at some time during their lives. In 2015 an estimated 3.1 million adults in the United States had received a diagnosis of Irritable Bowel Disease, and according to the Crohn's & Colitis Foundation, and Crohn's disease may affect as many as 780,000 Americans. Therefore, there remains a significant need for the treatment and management of gastrointestinal diseases and other disorders mediated by related biological pathways.

### SUMMARY OF THE INVENTION

Various embodiments include methods for treating a subject suffering from gastrointestinal radiation injury (GRI) or graft vs. host disease (GvHD), or a subject at risk of developing GRI or GvHD, comprising administering a therapeutically effective amount of LIF or variant thereof or a biologically active fragment of LIF or variant thereof to treat the subject. The active agent can be administered as an enteric formulation, or is formulated for topical administration to an area of the intestine. In some embodiment the active agent is formulated for targeted absorption by the small or large intestine. The therapeutic amount of LIF or a variant thereof, or a biologically active fragment of LIF or a variant thereof administered per day can be between 0.5 μg/kg and 100 μg/kg, between about 1 μg/kg and 75 μg/kg, between 0.25 μg/kg and 50 μg/kg, 5 μg/kg and 50 μg/kg, between 20 μg/kg and 40 μg/kg, or the amount is selected from the group consisting of 0.5 μg/kg, 1 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, and 60 μg/kg. In some embodiments, the active agent is administered once or twice per day for various numbers of days from 1 day to 1 month, until symptoms of the disease are gone, the subject no longer responds to treatment or the symptoms of the disease have reached an acceptable level. In preferred embodiments LIF is human LIF is identified by SEQ ID NO: 1, and the biologically active fragment of LIF is identified by SEQ ID NO:2, or is an active fragment comprising SEQ ID NO: 2.

In some of the present methods, the subject receives radiation treatment for cancer or as preparation for a bone marrow transplant, and the therapeutic amount LIF or variant thereof or a biologically active fragment of LIF or a variant thereof is administered between 1 to 2 days, 2 to 4 days, 7 days, 2 weeks or up to 1 month before the subject receives radiation treatment to reduce or otherwise ameliorate gastrointestinal radiation injury or GvHD. In an alternate embodiment, the active agent is administered once or twice daily for the duration of radiation treatment. In another embodiment, the active agent is once or twice daily for up to 45 days following the radiation treatment.

Another embodiment is directed to a method for maintaining or increasing intestinal epithelial cell (IEC) growth in isolated mammalian intestine tissue or in artificial intestine comprising mammalian ISC and IEC, comprising contacting the isolated or artificial intestine in vitro with LIF or a variant thereof, a biologically active fragment of LIF or a variant thereof in an amount that maintains or increases IEC growth. And another embodiment is directed to a method for treating a subject exposed to a damaging level of radiation, comprising administering a therapeutically effective amount of LIF or a variant thereof, a biologically active fragment of LIF or a variant thereof one or more times per day for as long as symptoms appear, which amount is between 0.5 μg/kg and 100 μg/kg, between about 1 μg/kg and 75 μg/kg, between 0.25 μg/kg and 50 μg/kg, 5 μg/kg and 50 μg/kg, between 20 μg/kg and 40 μg/kg, or the amount is selected from the group consisting of 0.5 μg/kg, 1 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, and 60 μg/kg.

Other embodiments are directed to pharmaceutical compositions formulated for topical application or for enteric absorption in the small or large intestine, comprising a pharmaceutically acceptable excipient and LIF or a variant thereof, or a biologically active fragment of LIF or a variant thereof in an amount between 0.5 μg/kg and 100 μg/kg, between about 1 μg/kg and 75 μg/kg, between 0.25 μg/kg and 50 μg/kg, 5 μg/kg and 50 μg/kg, between 20 μg/kg and 40 μg/kg, or the amount is selected from the group consisting of 0.5 μg/kg, 1 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, and 60 μg/kg. Fin a preferred embodiment, LIF or a variant thereof, or a biologically active fragment of LIF or a variant thereof is a recombinant human peptide, including wherein the recombinant human LIF peptide is identified by SEQ ID NO: 1, and the biologically active fragment of LIF is identified by SEQ ID NO:2, or is an active fragment comprising SEQ ID NO: 2.

Included in the embodiments are methods for reducing GvHD following a solid tumor transplant or blood transfusion, comprising administering a therapeutically effective amount of LIF or a variant thereof, a biologically active fragment of LIF or a variant thereof one or more times per day for as long as symptoms appear, which amount is between 0.5 μg/kg and 100 μg/kg, between about 1 μg/kg and 75 μg/kg, between 0.25 μg/kg and 50 μg/kg, 5 μg/kg and 50 μg/kg, between 20 μg/kg and 40 μg/kg, or the amount is selected from the group consisting of 0.5 μg/kg, 1 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, and 60 μg/kg.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1A through 1F. The expression of LIF in the intestinal epithelium. FIG. 1A & FIG. 1B. LIF expression in the intestinal crypts was determined by (FIG. 1A) IHC staining of the duodenum and ileum, and (FIG. 1B) IF staining of the small intestine of WT and LIF KO mice. FIG. 1C. LIF expression in the mouse colon was determined by IF staining. FIG. 1D. LIF expression in human colon tissues was determined by IHC staining. FIG. 1E & FIG. 1F. Co-localization of LIF with Olfm4 (FIG. 1E) and Lysozyme (FIG. 1F) was observed in the small intestine of WT mice as determined by IF staining. The white arrow represents the co-localization of LIF with Olfm4 (FIG. 1E) and Lysozyme (FIG. 1F), respectively.

FIG. 2A through FIG. 2I. LIF deficiency impairs the development of the small intestine in mice. FIG. 2A. Representative H&E staining images of the duodenum and ileum of WT and LIF KO mice. FIG. 2B. Quantification of the villus length (n=120 villi from at least 3 mice/group), villus density (n=48 fields from at least 3 mice/group), and crypt depth (n=120 crypts from at least 3 mice/group) in the small intestine of WT and LIF KO mice. The villus length and crypt depth were normalized to the average weight of WT mice at the same age with the same gender. FIG. 2C. A significantly decreased number and percentage of proliferating cells in the crypts of LIF KO mice. Left panels: representative images of IHC staining of Ki67. Right panels: quantification of the percentage and number of Ki67 positive (Ki67⁺) cells/crypt. n=120 crypts from at least 3 mice/group. FIG. 2D-FIG. 2G. The number of ISCs was greatly reduced in LIF KO mice compared with WT mice. d. The mRNA level of Olfm4 in the small intestine of mice was determined by quantitative real-time PCR assays and normalized with β-actin. n=6 mice/group. FIG. 2E. IHC staining of Olfm4 in the duodenum and ileum of mice. FIG. 2F. The percentage of Lgr5-GFP⁺ cells in small intestinal epithelial cells was decreased in LIF KO; Lgr5-GFP mice compared with Lgr5-GFP⁺ WT mice. Left panels: representative images of flow cytometry analysis of Lgr5-GFP⁺ cells. Right panel: quantification of the percentage of Lgr5-GFP⁺ cells in small intestinal epithelial cells. n=5 mice/group. FIG. 2G. IF staining of GFP in the duodenum and ileum of WT; Lgr5-GFP and LIF KO; Lgr5-GFP mice. FIG. 2H & FIG. 2I. The number of Paneth cells was greatly reduced in LIF KO mice. FIG. 2H. The mRNA level of Lysozyme in the small intestine of mice. n=6/group. FIG. 2I. IHC staining of Lysozyme in the duodenum and ileum of mice. In FIG. 2B, FIG. 2C, FIG. 2D, FIG. 2F & FIG. 2H, data are presented as mean±SD. **: p<0.01, ***: p<0.001; Student's t-test.

FIG. 3A through FIG. 3F. LIF deficiency impairs the growth of intestinal organoids. FIG. 3A. Representative images showing the growth of intestinal organoids from WT and LIF KO mice. For LIF treatment, LIF (50 ng/mL) was added into the medium. FIG. 3B. Quantification of the surface area of the intestinal organoids. Data are presented as mean±SD. n≥30/group, **: p<0.01; One-way ANOVA followed by SNK test. FIG. 3C. Quantification of the percentage of organoid formation. Organoids, organoids 1, organoids 2, and organoids 3+ refers to organoids with no bud, one bud, two buds and three or more buds, respectively. n≥100/group, *: p<0.05; **: p<0.01; Fisher's exact test. FIG. 3D. IF staining of Ki67 in the intestinal organoids. FIG. 3E. IF staining of GFP in the intestinal organoids from WT; Lgr5-GFP and LIF KO; Lgr5-GFP mice. FIG. 3F. Intestinal organoids derived from LIF KO mice showed significantly reduced ability to regenerate new villus and crypt structures to generate complete organoids upon passaging (P). Top panel: quantification of the percentage of budded organoids after passaging. Data are presented as mean±SD. n=4/group. **: p<0.01; ***: p<0.001; Student's t-test. Bottom panels: representative images showing the growth of organoids 3 days after each passaging.

FIG. 4A through FIG. 4D. LIF deficiency inhibits the β-catenin signaling pathway in the small intestine of mice. FIG. 4A. The mouse small intestine of LIF KO mice had significantly less nuclear β-catenin staining compared with WT mice. Left panels: IHC staining of β-catenin in the small intestine. Right panels: Quantification of the number (left) and percentage (right) of cells with positive nuclear β-catenin staining/crypt. n=120 crypts from at least 3 mice/group. FIG. 4B & FIG. 4C. Relative mRNA expression levels of a group of well-known β-catenin target genes, including Axin2, Ascl1, and Lgr5, in the small intestine (FIG. 4B) and colon (FIG. 4C) from WT and LIF KO mice. FIG. 4D. Relative mRNA expression levels of Axin2, Ascl1, Olfm4 and Lgr5 in the organoids from WT and LIF KO mice. For FIG. 4B-FIG. 4D, mRNA levels were measured by quantitative real-time PCR assays and normalized with β-actin. n=6 mice/group. Data are presented as mean±SD. *: p<0.05, **: p<0.01, ***: p<0.001; Student's t-test.

FIG. 5A through FIG. 5I. LIF upregulates the β-catenin signaling via AKT/GSK3β in the small intestine. FIG. 5A. A schematic model depicting the stabilization of β-catenin through the AKT and Wnt signaling pathways. FIG. 5B. Decreased phosphorylation levels of AKT Ser-473 (p-AKT) and GSK3β Ser-9 (p-GSK3β) in the small intestine of LIF KO mice as determined by Western-blot assays. FIG. 5C & FIG. 5D. The PI3K/AKT inhibitor Wortmannin and AKT inhibitor Capivasertib inhibited the growth and proliferation of WT organoids and LIF KO organoids supplemented with recombinant LIF, but had a much less pronounced effect on LIF KO organoids. FIG. 5C. Top panels: representative images showing the growth of WT, LIF KO, and LIF KO+LIF (50 ng/ml) intestinal organoids with or without Wortmanin (1 μM) or Capivasertib (1 μM) treatment. Bottom panel: quantifications of the surface area of organoids. FIG. 5D. IF staining of Ki67 in WT, LIF KO and LIF KO+LIF intestinal organoids with or without Wortmanin or Capivasertib treatment at day 6. FIG. 5E & FIG. 5F. The AKT agonist SC79 significantly enhanced the growth and proliferation of LIF KO organoids. FIG. 5E. Top panels: representative images showing the growth of WT and LIF KO intestinal organoids with or without LIF (50 ng/ml) or SC79 (5 μM) treatment. Bottom panel: quantifications of the surface area of organoids. FIG. 5F. IF staining of Ki67 in WT and LIF KO intestinal organoids with or without LIF or SC79 treatment at day 6. FIG. 5G. Relative mRNA expression levels of Axin2, Ascl1, Olfm4 and Lgr5 in WT and LIF KO intestinal organoids with or without Capivasertib or SC79 treatment. mRNA levels were measured by quantitative real-time PCR assays and normalized with β-actin. FIG. 5H & FIG. 5I. The GSK3β inhibitor CHIR99021 rescued the impaired growth and proliferation of LIF KO organoids. FIG. 5H. Top panels: representative images showing the growth of WT and LIF KO intestinal organoids with or without CHIR99021 treatment (3 μM). Bottom panel: quantifications of the surface area of organoids. FIG. 5I. IF staining of Ki67 in WT and LIF KO intestinal organoids with or without CHIR99021 treatment at day 6. In FIG. 5C, FIG. 5E, FIG. 5G & FIG. 5H, data are presented as mean±SD. *: p<0.05; **: p<0.01; ***: p<0.001; One-way ANOVA followed by SNK test. For FIG. 5C, FIG. 5E & FIG. 5H, n≥30/group; for FIG. 5G, n=4/group.

FIG. 6A through FIG. 6F. LIF deficiency impairs regeneration of the intestinal epithelium after IR which can be rescued by administering recombinant mouse LIF. FIG. 6A. The morphology of the duodenum and ileum of WT, LIF KO, and LIF KO mice injected with recombinant mouse LIF (LIF KO+LIF) as examined by H&E staining at 72 h after 12 Gy whole-body IR. Mice were irradiated with 12 Gy whole-body IR on day 0, and injected with recombinant LIF (i.p.; 30 ng/g body weight) or vehicle (PBS) twice a day from day −3 to day 3. Left panel: a schematic diagram of experimental procedures; right panels: representative H&E images. FIG. 6B & FIG. 6C. A significantly decreased number of proliferating crypts in LIF KO mice at 72 h post IR, which was rescued by LIF injection. FIG. 6B. Representative images of IHC staining of Ki67 in the duodenum and ileum of mice post IR. FIG. 6C. Quantification of viable crypts/field in the duodenum and ileum of mice post IR. A viable crypt is defined as a crypt-like structure containing at least five adjacent Ki67⁺ cells. FIG. 6D & FIG. 6E. A significantly reduced number of ISCs in LIF KO mice at 72 h post IR, which was rescued by LIF injection. FIG. 6D. Representative images of IHC staining of Olfm4 in the duodenum and ileum of mice post IR. FIG. 6E. Quantification of Olfm4 positive crypts/field in the duodenum and ileum of mice post IR. FIG. 6F. Kaplan-Meier survival curve of WT, LIF KO, and LIF KO+LIF mice post 12 Gy IR. In FIG. 6C & FIG. 6E, data are presented as mean±SD. n=30 fields from at least 3 mice/group. ***: p<0.001; Student's t-test. In FIG. 6F, ***: p<0.001; Kaplan-Meier survival analysis.

FIG. 7A through FIG. 7F. Administering recombinant mouse LIF promotes regeneration of the intestinal epithelium and prolongs survival of WT mice after IR. FIG. 7A. Supplementing the medium with recombinant mouse LIF (50 ng/ml) promoted the growth of organoids from WT mice. Left panel: representative images showing the growth of intestinal organoids. Right panel: quantification of the surface area of the intestinal organoids. FIG. 7B. The morphology of the duodenum and ileum of WT mice with or without injection of recombinant LIF (WT+LIF) as examined by H&E staining at 72 h after 12 Gy IR. FIG. 7C. IHC staining of Ki67 in the duodenum and ileum of WT and WT+LIF mice at 72 h after 12 Gy IR. Left panel: representative images of Ki67 IHC of the duodenum and ileum. Middle panel, quantification of crypt length. Right panel: the number of Ki67⁺ cells/crypt. Data are presented as mean±SD. n=120 crypts from at least 3 mice/group. FIG. 7D & FIG. 7E. Quantification of the number of viable crypts/field (FIG. 7D) and Olfm4 positive crypts/field (FIG. 7E) in the duodenum and ileum of WT and WT+LIF mice at 72 h post 12Gy IR. FIG. 7F. K aplan-Meier survival curve of WT and WT+LIF mice post 12 Gy whole-body IR (left) and 9 Gy whole-body IR (right). In FIG. 7A, FIG. 7D, & FIG. 7E, data are presented as mean±SD. n=30 fields from at least 3 mice/group. *: p<0.05; **: p<0.01; ***: p<0.001; Student's t-test. In FIG. 7F, *: p<0.05; **: p<0.01; Kaplan-Meier survival analysis.

FIG. 8 LIF overexpression promotes the regeneration of intestinal epithelium after irradiation in inducible LIF-tgflox/+/Cre-ERT2 mice. Left top panel shows the treatment of tamoxifen and irradiation in LIF-tgflox/+/Cre-ERT2 mice. Right panels: representative IHC images of Ki67 staining in intestine tissues of mice with or without LIF overexpression at 72 h after irradiation. Left lower panels: Quantification of numbers of viable crypts per field in the intestine tissues of mice with or without LIF overexpression at 72 h after irradiation. **: p<0.01, Student's t-test.

FIG. 9 Graft versus host disease (GvHD) was established using a MHC haplo-mismatch transplantation mouse model. After pre-conditioning with ionized radiation, recipient mice received bone marrow (BM) transplantation with or without allogeneic donor T cells. While recipient mice receiving BM transplantation without T cells appeared normal throughout lifespan, those receiving allogeneic donor T cells (BM-T cell) developed GvHD and had a reduced life span (medium life span of ˜30 days). Administering LIF to mice receiving BM-T cells greatly reduced the development of GvHD and prolonged the survival of mice.

FIG. 10A through FIG. 10B. Blocking STAT3 or MAP signaling pathway does not have a significant effect on the growth and proliferation of WT or LIF KO organoids. Quantifications of the surface area (FIG. 10A) and the percentage of intestinal organoids formation (FIG. 10B) of WT and LIF KO intestinal organoids with or without treatments of Wortmannin (1 μM), Stattic (STAT3 inhibitor, 2 μM), or SB242235 (MAPK inhibitor, 1 μM). In a, data are presented as mean±SD. n≥30/group, *: p<0.05; ***: p<0.001; One-way ANOVA followed by SNK test. In b, n≥100/group, **: p<0.01; ***: p<0.001; Fisher's exact test.

### DETAILED DESCRIPTION

### 1. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar technical references.

Abbreviations used herein include: intestinal stem cell (ISC), intestinal epithelial cell (IEC), Transit-amplifying cells (TA cells), crypt base columnar cells (CBC cells).

As used herein, “a” or “an” may mean one or more than one. As used herein when used in conjunction with the word “comprising,” the words “a” or “an” may mean one or more than one. As used herein “another” may mean at least a second or more. Unless otherwise required by context, singular terms include pluralities and plural terms include the singular.

The term “about,” as used herein, means plus or minus 20 percent of the recited value, so that, for example, “about 0.125” means 0.125±0.025, and “about 1.0” means 1.0±0.2. In some instances, the term “about” may include numerical values that are rounded to the nearest significant figure.

As used herein, the terms “treat”, “treating” and “treatment” include improving, reducing, or eliminating a disease or condition or a symptom of a disease or condition; slowing or reversing the progression of a disease or condition or a symptom of a disease or condition; decreasing the severity and/or frequency of the occurrence of a disease or condition or a symptom of a disease or condition; and preventing, lessening the severity of, or lessening the chance of the occurrence or recurrence of a disease or condition or a symptom of a disease or condition.

In general, treatment refers to ameliorating, blocking, reducing, decreasing or inhibiting a disease condition or symptom by about 1% to about 100% compared to a subject to which the peptides and/or compositions of the present invention have not been administered. Preferably, the ameliorating, blocking, reducing, decreasing or inhibiting is about 100%, 99%, 98%, 97%, 96%, 95%, 90%, 80%, 70%, 60%, 50%, 40%, 30%, 20%, 10%, 5% or 1% compared a subject to which the peptides or compositions have not been administered.

The term “active fragment” or “active variant” refers to a fragment or variant of a sequence that maintains its biological activity. In the example of LIF, active fragments or active variants of LIF maintain their ability to bind to LIF receptor (LIFR).

Leucine-rich repeat-containing G-protein coupled receptor 5 (LGR5) also known as G-protein coupled receptor 49 (GPR49) or G-protein coupled receptor 67 (GPR67), is a protein that in humans is encoded by the LGR5 gene. ^{[5][6]} It is a member of GPCR class A receptor proteins. R-spondin proteins are the biological ligands of LGR5. LGR5 is expressed across a diverse range of tissue such as in the muscle, placenta, spinal cord and brain and particularly as a biomarker of adult stem cells in certain tissues.

The term “co-administration,” “administered in combination with,” and their grammatical equivalents, as used herein, encompasses administration of two or more agents to a subject so that both agents and/or their metabolites are present in the subject at the same time. Co-administration includes simultaneous administration in separate compositions, administration at different times in separate compositions, or administration in a composition in which both agents are present.

As used herein, cytokine leukemia inhibitory factor (LIF) (Synonyms:HILDA) refers to a multi-function interleukin 6 class cytokine that affects cell growth by inhibiting differentiation and has displays pleiotropic effects on various cell types and organs. LIF binds to LIF receptor (LIFK-α) which forms a heterodimer with a specific subunit common to all members of that family of receptors, the GP130 signal transducing subunit to activate several signaling pathways, including the JAK/STAT, MAPK, and PI3-K pathways, in a cell and tissue specific manner, to exert its function. (Human canonical LIF Primary accession number: P15018, herein SEQ ID NO: 1.) It has now been discovered that LIF also maintains β-catenin activity through the AKT/GSK3β signaling to regulate intestinal stem cell (ISC) function. These results reveal a previously unidentified and crucial role of LIF in ensuring ISC function, and demonstrate that LIF regulates the function of adult stem cells. Unless specified otherwise, LIF may pertain to the natural LIF sequence (e.g. SEQ ID NO:1) or active fragments or active variants thereof.

Human LIF canonical sequence, Isoform A,
Accession number P15018 ISOFORM A)
SEQ ID NO: 1
MKVLAAGVVPLLLVLHWKHGAGSPLPITPVNATCAIRHPCHNNLMNQIRSQ
LAQLNGSANALFILYYTAQGEPFPNNLDKLCGPNVTDFPPFHANGTEKAKL
VELYRIVVYLGTSLGNITRDQKILNPSALSLHSKLNATADILRGLLSNVLC
RLCSKYHVGHVDVTYGPDTSGKDVFQKKKLGCQLLGKYKQIIAVLAQAF

The term “effective amount” or “therapeutically effective amount” as used herein means that the amount of one or more agent, material or composition comprising one or more agents as described herein which is effective for producing the desired effect in a subject; for example, an amount of the compositions described herein effective to promote renewal of intestinal stem cells (ISC), or maintenance and regeneration of IEC and ISC. Effective amount also pertains to an amount that reduces or ameliorates one or more symptoms of a disease, including but not limited to gastrointestinal radiation injury or GVHD, inflammatory bowel disease like colitis, ulcerative colitis, Chron's disease.

Enteral formulation as used herein means administration routes comprising a non-local administration of the drug via the GI digestive tract. Enteral administration involves the esophagus, stomach, and small and large intestines (i.e., the gastrointestinal tract). Methods of administration include oral, sublingual (dissolving the drug under the tongue), and rectal.

An “enumerated agent” or “active agent” or “active drug” is an agent/drug selected from the group human LIF and biologically active fragments or variants of LIF thereof, and includes naturally occurring LIF and recombinant peptides. If using an enumerated agent to treat a non-human animal including veterinary animals, the LIF may have a non-human sequence.

The term “Gastrointestinal radiation injury (GRI)” as used herein means the collective symptoms of radiation damage to the GI tract caused by ionizing radiation for any reason, including administration as a treatment for different types of cancers, bone marrow transplant or accidental exposure to a deleterious amount of radiation. The TD5/5 and TD50/5 doses for one third of small bowel irradiation were estimated at 50 Gy and 60 Gy respectively. The TD5/5 and TD50/5 for the whole-organ irradiation were 40 Gy and 55 Gy respectively. TD5/5: Radiation dose associated with 5% of patients' risk of delayed toxicity in 5 years; TD50/5: The radiation dose associated with 50% of patients' risk of delayed toxicity in 5 years. (Shadad A, Sullivan F., et al. World J. Gastroenterol, 19:185).

GI syndrome as used herein means a fatal syndrome involving diarrhea, bacterial translocation, and hemorrhage occurs when large areas of the intestine are irradiated.

The term “Graft-versus-host disease (GvHD)” as used herein is a medical complication that can occur following the receipt of transplanted tissue from a genetically different person. GvHD is commonly associated with stem cell transplants such as those that occur with bone marrow transplants. GvHD also applies to other forms of transplanted tissues such as solid organ transplants. White blood T-cells (herein T-cells) of the donor's immune system that remain within the donated tissue (the graft) recognize the recipient (the host) as foreign (non-self). The white blood T-cells present within the transplanted tissue then attack the recipient's own cells, which leads to GvHD. This should not be confused with a transplant rejection that occurs when the immune system of the transplant recipient rejects the transplanted tissue. By contrast, GvHD occurs when the donor's immune system's white blood cells reject cells in the recipient. The underlying principle of alloimmunity is the same, but the details and course differ. GvHD can also occur after a blood transfusion if the blood products retain donor T-cells either because the blood was not irradiated or treated with an approved pathogen-reduction system prior to infusion into the recipient. Acute GvHD of the GI tract can result in severe intestinal inflammation, sloughing of the mucosal membrane, severe diarrhea, abdominal pain, nausea, and vomiting. This is typically diagnosed via intestinal biopsy.

The term “Inflammatory bowel disease (IBD)” as used herein is a group of inflammatory conditions of the colon and small intestine. Crohn's disease and ulcerative colitis are the principal types of inflammatory bowel disease. Crohn's disease affects the small intestine and large intestine, as well as the mouth, esophagus, stomach and the anus, whereas ulcerative colitis primarily affects the colon and the rectum. In some cases, Crohn's affects only the colon, it's called Crohn's granulomatous colitis. This type of Crohn's causes diarrhea and rectal bleeding. People may develop abscesses and ulcers in the area of the anus. Other symptoms include joint pain and skin lesions. For the purpose of this invention, both of these are collectively referred to as Crohn's disease.

The term “Intestinal crypt,” as used herein also refers to as an intestinal gland or crypt of Lieberkühn, is a found in the intestinal epithelium lining of the small intestine, including the duodenum, jejunum and ileum, and large intestine (colon), where they are sometimes called colonic crypts. The crypts and intestinal villi are covered by epithelium, which contains multiple types of cells: enterocytes (absorbing water and electrolytes), goblet cells (secreting mucus), enteroendocrine cells (secreting hormones), cup cells, tuft cells, and at the base of the crypt, Paneth cells (secreting anti-microbial peptides) and stem cells. These cells are not all present in the colon. In the colon, crypts do not have Paneth cells.

The term “large intestine” as used herein includes the synonyms “the large bowel” and “colon,” is the last part of the gastrointestinal tract and of the digestive system in vertebrates. Water is absorbed here and the remaining waste material is stored as feces before being removed by defecation. Many times in the literature the large intestine and colon overlap in meaning whenever anatomic precision is not the focus. Most sources define the large intestine as the combination of the cecum, colon, rectum, and anal canal.

The term “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

The term “small intestine” or “small bowel” as used herein refers to the part of the gastrointestinal tract between the stomach and the large intestine, and is where most of the end absorption of food takes place. The small intestine has three distinct regions—the duodenum, jejunum, and ileum. The duodenum is the shortest part of the small intestine and is where preparation for absorption begins. It also receives bile and pancreatic juice through the pancreatic duct, controlled by the sphincter of Oddi. The primary function of the small intestine is the absorption of nutrients and minerals from food, using small finger-like protrusions called villi.

A “stem cell” means any precursor cell, whose daughter cells may differentiate into other cell types. In general, a stem cell is a cell capable of extensive proliferation, generating more stem cells (self-renewal) as well as more differentiated progeny, such as intestinal epithelial cells (IEC) in the context of the present invention. Thus, a single stem cell can generate a clone containing millions of differentiated cells such as IEC as well as a few stem cells. Stem cells thereby enable the continued proliferation of tissue precursors over a long period of time. Without being bound by theory, it is currently believed that stem cells exist in virtually every tissue in the adult body, and that such stem cells provide an endogenous mechanism for some level of repair in adult tissues. Stem cells are found in the intestine.

The terms “subject,” “individual” or “patient” are used interchangeably herein, and refer to a vertebrate, for example a mammal, including a human Mammals include, but are not limited to, murines, simians, humans, farm animals, sport animals, and pets. Tissues, cells and their progeny of a biological entity obtained in vitro or cultured in vitro are also encompassed.

The term “susceptibility” or “susceptible” as used herein, refers to a subject determined to be at risk for having a disease condition. Such a determination may be based on an analysis including, but not limited to, (i) familial disease history, (ii) a genotypic characteristic of the subject, and/or (iii) a phenotypic characteristic of the subject.

The phrases “systemic administration,” “administered systemically,” “peripheral administration” and “administered peripherally” as used herein mean the administration of a compound such that it enters the animal's system and, thus, is subject to metabolism and other like processes, for example, subcutaneous administration.

A “therapeutic effect” as used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof. A prophylactic amount is an amount that achieves this result.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication, amelioration or stabilization of the underlying disorder being treated (e.g., in the context of many gastrointestinal diseases, complete or substantially complete mucosal healing). Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. For purposes of this invention, beneficial or desired clinical results include, but are not limited to, one or more of the following: reducing, decreasing symptoms resulting from the disease, increasing the quality of life of those suffering from the disease, decreasing the dose of other medications required to treat the disease, enhancing the effect of another medication, delaying the progression of the disease, and/or prolonging survival of individuals. Treatment includes preventing the disease, that is, causing the clinical symptoms of the disease not to develop by administration of a protective composition prior to the induction of the disease, suppressing the disease, that is, causing the clinical symptoms of the disease not to develop by administration of a protective composition after the inductive event but prior to the clinical appearance or reappearance of the disease, inhibiting the disease, that is, arresting the development of clinical symptoms by administration of a protective composition after their initial appearance, preventing re-occurring of the disease and/or relieving the disease, that is, causing the regression of clinical symptoms by administration of a protective composition after their initial appearance.

### 2. Embodiments of the Invention

It has now been discovered that LIF plays an essential role in maintaining Intestinal Stem Cell (ISC) function in self-renewal and proliferation, and in turn in maintaining and stimulating (inducing or otherwise increasing) intestinal epithelial cell (IEC) regeneration in subjects that have been exposed to deleterious levels of ionizing radiation or have developed GvHD. LIF is expressed in the ISC niche, including ISCs, TA cells and Paneth cells, in the intestinal tissues in both mice and humans. It has been discovered that LIF maintains the function of ISCs by up-regulating β-catenin activity mainly through the AKT/GSK3β signaling. It is shown herein that LIF deficiency in mice caused both impaired homeostasis of intestinal epithelium at physiological conditions and impaired regeneration of intestinal epithelium after injury in response to whole body radiation (IR) with 12 GRAY, and further that administering recombinant LIF protein rescued this impairment and restored the regeneration of intestinal epithelium after whole body IR injury in mice with LIF deficiency (LIF KO). Further, recombinant LIF protein showed a protective role in wild-type (WT) mice through promotion of intestinal epithelial regeneration after IR radiation, which in turn protected the mice from developing radiation-induced gastrointestinal (GI) syndrome.

Additional discoveries include the ability of LIF to ameliorate or reduce the severity of GvHD following autologous or non-autologous transplants.

Results from this study thus show a crucial and a previously unrecognized role of LIF in supporting ISC function, promoting regeneration of intestinal epithelium in response to radiation injury, and protecting against radiation-induced GI syndrome and GvHD.

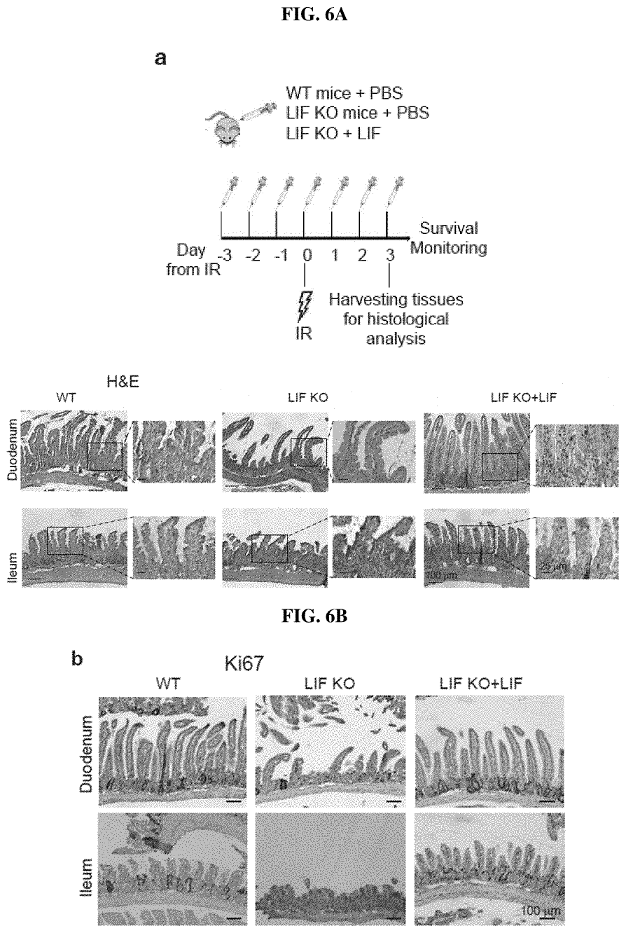

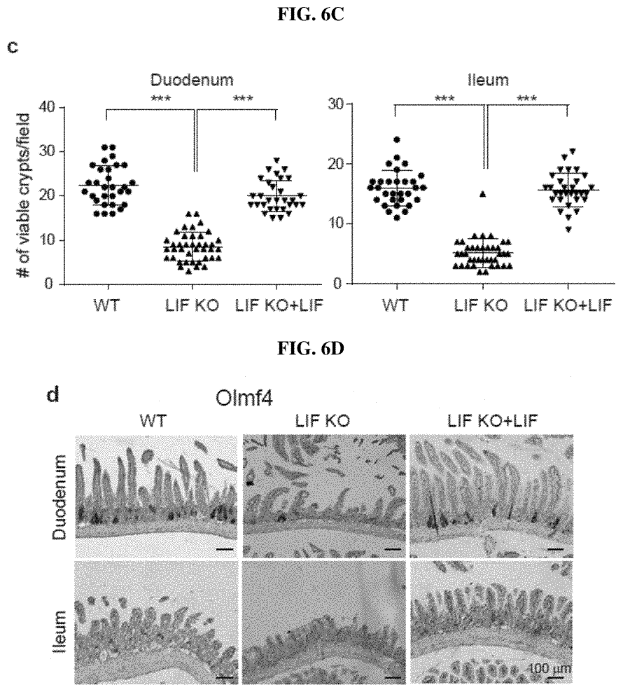

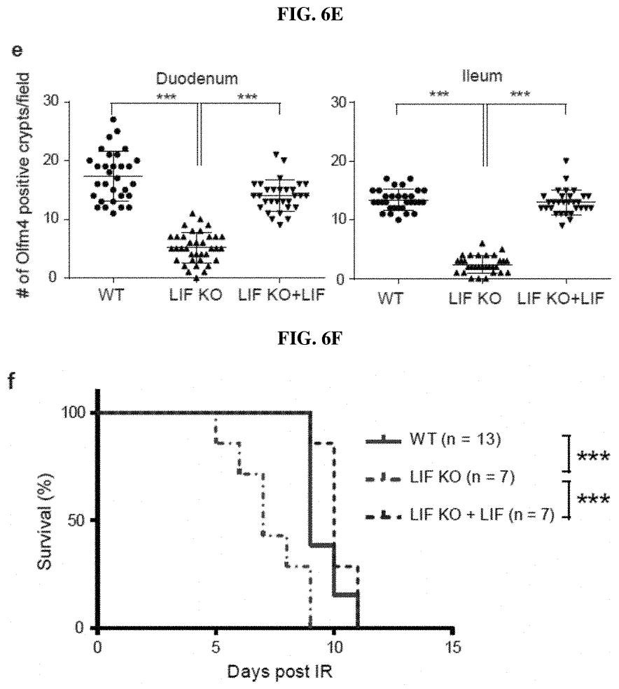

LIF deficiency in mice impaired both renewal of intestinal epithelium under physiological conditions and regeneration of intestinal epithelium after injury in response to ionizing radiation (IR) 12 Gy whole-body irradiation. A significant reduction of about 50-70% in the number of viable crypts, defined as containing at least five adjacent Ki67⁺ cells within a crypt-like structure, were observed in the small intestine (ileum and duodenum) of LIF KO mice compared with the small intestine of WT mice 72 h after whole body IR with 12 GRAY (See FIG. 6C). Further, both LIF overexpression in inducible LIF transgenic (LIF-tg^{flox/+}/Cre-ERT2) mice and administration of recombinant LIF to wild type mice resulted in significantly higher intestinal crypt regeneration, compared to controls following ionizing radiation, prolonged survival and improved weight gain.

Mechanistically, LIF maintained the ability of ISCs in intestinal epithelial to maintain cell renewal after IR through AKT/GSK3β signaling to up-regulate β-catenin activity, which in turn maintained normal rates of cell renewal The results show a crucial and a previously unrecognized role of LIF in ensuring ISC function in intestinal epithelial cell maintenance and regeneration under physiologic conditions and after whole body irradiation to mitigate radiation injury including GvHD. Certain embodiments relate to the therapeutic use of LIF and variants thereof, biologically active fragments and variants thereof (collectively active agents) to treat gastrointestinal radiation injury (GRI) (also herein referred to as RI/radiation injury) and GvHD, by promoting ISC function in self-renewal and proliferation, and in turn in maintaining and stimulating (inducing or otherwise increasing) intestinal epithelial cell (IEC) regeneration through the beta-catenin pathway. Routes of administration, effective doses, treatment regimens, and pharmaceutical formulations are discussed below. Certain embodiments are directed to methods for treating gastrointestinal radiation injury (GRI), for example in a cancer patient receiving radiation therapy or in a BMT recipient who has received or will receive radiation to eradicate cancer cells in the marrow, with a therapeutically effective amount of an active agent before during or after ionizing radiation to minimize IEC damage and cell loss.

It is not only radiation that causes damage in patients undergoing BMT. The transplanted bone marrow used in a nonautologous transplant will have heterologous, foreign T cells that also induce intestinal damage causing a GI disorder. It has also been discovered that pretreatment of a subject with LIF or other active agent prior to (administration can also be continued following)g a nonautologous bone marrow transplant with allogenic T cells greatly reduced the severity of GvHD and prolonged survival in mice using an MHC haplo-mismatch transplantation model.

Certain embodiments are also directed to a method for treating a subject exposed accidentally to a damaging level of radiation by administering a therapeutic dose of LIF to reduce the symptoms of radiation-induced GI damage. LIF or other active agent can be administered during the course of an enumerated GI disease, and after symptoms have subsided to maintain intestinal health. Therapeutically effective amounts of an active agent can be administered prior to irradiation or bone marrow transplant to reduce or ameliorate the symptoms of radiation injury or GvHD can be administered prior to whole body irradiation whether for cancer treatment prior to a bone marrow transplant such as with allogenic T cells.

Based on these observations, certain other embodiments of the invention include methods for maintaining or increasing the number of healthy intestinal epithelial cells (IEC) (i.e. maintain/increase IEC number or IEC regeneration) in isolated intestine tissue or in artificial intestine comprising ISC and IEC, comprising contacting the isolated or artificial intestine in vitro with LIF or a variant thereof, a biologically active fragment of LIF or a variant thereof in an amount that maintains or increases IEC growth. This amount also reduces or prevents ISC or IEC damage or decreased numbers of ISC or IEC. The isolated mammalian intestinal tissue may be intended for an autologous or a nonautologous transplant into a mammal in need of such treatment, or be used for research purposes.

Other GI diseases and disorders that can be treated with the methods, pharmaceutical formulations and active agents of the present invention include inflammatory bowel disease, colitis, ulcerative colitis, and Chron's disease. Ulcerative colitis is a disease of the colon wherein it is ulcerated and a symptomatic patient may have diarrhea. UC is treated as an autoimmune disease with anti-inflammatory or immunosuppressive agents, including biological therapeutics that targeting specific components of the immune response. However, not all patients respond to such treatment.

New treatment options for intestinal diseases are needed.

Certain embodiments are directed to pharmaceutical formulations of LIF and other active agents for enteric administration or for topical administration to the intestine or application to isolated intestinal cells, explants or artificial intestine.

In the following description, for the purposes of explanation, numerous specific details are set forth in order to provide a thorough understanding of the present invention. It will be apparent, however, to one skilled in the art that the present invention may be practiced without these specific details.

LIF and other active agents described here are expected to be safe when administered locally (enteric administration or topical administration) and transiently to the intestine, even when there is long term use. By transient here we mean that LIF is not released constantly over a long period of time, but intermittently. Due to the quick turn-over rate of intestinal epithelium, LIF can be administered before or during the injury to prevent the damage and/or promote the regeneration of intestinal epithelium.

### Overview

LIF is a multi-function cytokine that displays pleiotropic effects on various cell types and organs. Previously, we found that LIF is a target gene of tumor suppressor p53, and mediates the function of p53 in regulation of embryonic implantation in mice and humans (3, 4). LIF binds to its heterodimer receptor complex composed of LIF receptor (LIFK) and glycoprotein gp130 to activate several signaling pathways, including the JAK/STAT, MAPK, and PI3-K pathways, in a cell and tissue specific manner, to exert its function [2-4]. It is well-established that LIF plays a crucial role in maintaining the pluripotency of mouse embryonic stem cells (mESCs) (6, 7). However, basal LIF expression levels are generally low in normal adult tissues and the role of LIF in adult stem cells in somatic tissues is not well-understood. In mice, LIF is expressed at a very low level in the intestinal epithelium in the embryos, and is expressed in the crypt of the intestine after birth, which can be detected as early as P7. The expression of LIF is observed in several types of cells in the intestinal crypt, including ISCs, TA cells and Paneth cells, throughout the intestine in mice. The expression of LIF is also detected in the crypt in human colon tissues, suggesting that LIF has a conserved function in regulating ISCs in mice and humans.

The ISC niche is a complex cellular structure that plays a key role in stem cell maintenance, proliferation and differentiation. The ISC niche is likely to comprise several different cell types, each of which contributes cell-associated ligands and chemokines, soluble cytokines and growth factors that regulate stem cell behavior. (Sailaja B., He X., Li L, The regulatory niche for intestinal stem cells. J Physiol., 2016, 594:4827).

Adult intestinal stem cells reside in intestinal crypts that supply a special microenvironment that provides a unique signaling environment to regulate the balance between stem cell self-renewal and tissue regeneration to maintain tissue homeostasis (Moore & Lemischka, 2006). Moore, K. A. & Lemischka, I. R. Stem cells and their niches, Science 311, 1880-1885 (2006). The intestinal crypt is one of the best-defined adult stem cell models, which drives rapid self-renewal of the intestinal epithelium (Clevers, 2013) Clevers, H. The intestinal crypt, a prototype stem cell compartment. Cell 154, 274-284 (2013). Intestinal crypts contain leucine-rich repeat-containing G-protein coupled receptor 5 (Lgr5⁺) intestinal stem cells (ISCs), their transit-amplifying (TA) daughter cells and terminally differentiated Paneth cells. Lgr5⁺ is a marker for intestinal stein cells that are located at the bottom of the crypt. Under physiological conditions, ISCs generate precursors of enterocytes and secretory cells that divide and differentiate into enterocytes, goblet cells, enteroendocrine cells and tuft cells. The high turnover rate of the intestinal lining is due to a dedicated population of ISCs found at the base of the intestinal crypt. In the small intestines, these LGR5^{+ve} crypt base columnar cells (CBC cells) have broad basal surfaces and very little cytoplasm and organelles and are located interspersed among the terminally differentiated Paneth cells. These CBC cells generate the plethora of functional cells in the intestinal tissue: Paneth cells, enteroendocrine cells, goblet cells, tuft cells, columnar cells and the M cells over an adult's entire lifetime. Similarly, Lgr5⁺ expression in the colon resembles faithfully that of the small intestine.

Upon injury, such as by ionizing radiation, intestinal crypts typically display a remarkable regenerative capacity driven by ISCs and progenitor cells that have a remarkable plasticity in their capability to drive regeneration of the intestinal epithelium. In response to certain high doses of IR, the intestinal epithelium goes through an apoptotic phase with an increase in crypt cell apoptosis, a massive loss of crypt cells and a shortening of crypts and villi in the first couple days, which is followed by a proliferative phase showing regeneration of crypts by surviving crypt cells (10). Due to the interruption of the extremely rapid cell turnover and lose of sufficient crypts in the intestine, proper intestinal mucosal barrier can no longer be maintained, which is susceptible to infection. Eventually the intestine becomes ulcerated, a fatal phenomena called GI syndrome (40).

Gastrointestinal radiation injury (GRI): Radiation therapy can be used to treat any abdominal cancer or as preparation for a bone marrow transplants (BMT) to kill cancer cells in the bone marrow, and this radiation will impact the intestine causing damage by reducing ISC and IEC regeneration thereby making the patient vulnerable to gastrointestinal radiation injury (GRI). For pancreatic cancer, GI toxicity is a limiting factor in radiation therapy. The side effects associated with radiation include destruction of normal cells, especially the dividing cells which leads to GI damage that is reflected by early histological changes, functional alterations and symptoms of nausea, vomiting and diarrhea. These side effects often cause morbidity and may in some cases lower the efficacy of radiotherapy treatment because it limits the dose of radiation that can be used to control cancer. The collective symptoms of radiation damage to the GI tract are referred to herein as gastrointestinal radiation injury radiation injury (RI).

A number of signaling pathways are involved in the regulation of ISC function. It has been shown that Wnt/β-catenin signaling is crucial for normal stem cell function in the intestinal epithelium. In the absence of Wnt ligands, β-catenin protein is targeted by a multi-protein degradation complex, including Axin, APC, CK-1 and GSK3β, for phosphorylation and subsequent proteasomal degradation, thereby maintaining low baseline cytosolic levels of β-catenin. The binding of Wnt ligands to frizzled (Fzd) receptors inhibits β-catenin phosphorylation and degradation, which leads to β-catenin accumulation and nuclear translocation. In the nucleus, β-catenin interacts with TCF/LEF transcription factors to induce a panel of β-catenin target genes, whose products play essential roles for stem cell self-renewal and homeostasis of epithelial tissues. However, a recent study reported that Wnt ligands alone are insufficient to induce ISC self-renewal and expansion beyond a certain threshold (Yan, K. S., 2017; Nature 545, 238).

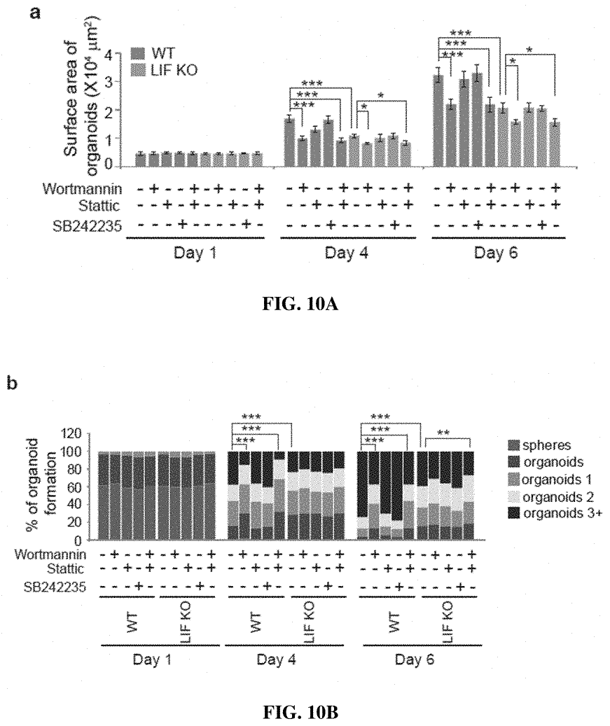

LIF exerts its effects through different signaling pathways. LIF can activate JAK/STAT3 signaling, which plays a central role in maintaining self-renewal and pluripotency of mouse embryonic stem cells (5, 46, 47). LIF can also activate MAPK signaling to mediate some of its functions. However, neither JAK/STAT3 nor MAPK pathway appears to play a major role in maintaining ISC functions; blocking STAT3 activity by Stattic showed a very limited effect FIG. 10, and blocking MAPK activity by SB242235 showed no obvious effect on ISC functions in WT organoids. These observations indicate that the pleiotropic functions of LIF are mediated by different downstream signaling pathways in a highly cell- and tissue-specific manner. It has been reported that LIF plays a protective role in mouse experimental colitis models (48), wherein microbiota dysregulation induces LIF secretion by intestinal epithelial cells (IECs) where it regulated the function of lamina propria lymphocytes via the STAT4 signaling and repair function of IECs via the STAT3/YAP signaling. However we found in contrast to this, that blocking STAT3 activity by Stattic had a very limited effect on IEC repair (FIG. 10). By contrast as discussed herein, we found that a different mechanism is responsible for ISC regeneration in animals suffering from radiation damage, rather than inflammation or autoimmune disease. GRI and GvHD, both involve subjects that have received deleterious levels of radiation which causes different damage than inflammation. LIF (and other active agents) regulate the function of ISCs specifically by upregulating β-catenin activity mainly through the AKT/GSK3 thereby increasing IEC regeneration. We have identified a crucial new role of LIF in supporting ISC function that in turn promotes regeneration of intestinal epithelium in response to radiation injury or GvHD, thus providing a treatment for GRI and to avoid developing gastric syndrome, where LIF acted unexpectedly by increasing beta-catenin activity. LIF is also useful to treat GvHD in subjects that have received a bone marrow transplant following radiation or a solid tissue transplant which may not involve radiation.

LIF active agents are expected to be safe when administered locally (enteric administration or topical administration) and transiently to the intestine, even when there is long term use. By transient here we mean that LIF is not released constantly over a long period of time, but intermittently. Due to the quick turn-over rate of intestinal epithelium, LIF can be administered before or during the injury to prevent the damage and/or promote the regeneration of intestinal epithelium. It has been reported that LIF is overexpressed in some solid tumors, including breast cancer, nasopharyngeal carcinoma, colon cancer, rhabdomyosarcoma and pancreatic cancers, and that chronic LIF overexpression promoted tumor progression/metastasis. (Ref, Liu S C, et al. Leukemia inhibitory factor promotes nasopharyngeal carcinoma progression and radioresistance. J Clin Invest. 2013; 123:5269-5283. Yu H, et al. LIF negatively regulates tumor-suppressor p53 through Stat3/ID1/MD.M2 in colorectal cancers. Nat Commun. 2014; 5:5218. Wysoczynski M, et al. Leukemia inhibitory factor: a newly identified metastatic factor in rhabdomyosarcomas. Cancer Res 2007 67(5):2131-40. Bressy C., et al. LIF drives neural remodeling in pancreatic cancer and offers a new candidate biomarker. Cancer Res., 2018; 78:909-921. Li X., et al., LIF promotes tumorigenesis and metastasis of breast cancer through the AKT-mTOR pathway. Oncotarget, 2014; 5:788-801.) However, there is no evidence showing that a transient temporary increase of LIF levels in normal tissues has tumorigenic effects. Under physiological condition, LIF is highly increased in uteri in tissues during embryo implantation.

A clinical trial also showed that recombinant human (rhLIF) was safely administered to treat patients with advanced cancers. The most common tumor types were non-small cell lung cancer and carcinoma of unknown primary. Recombinant human LIF had no adverse effects on blood progenitor cells, and hematopoietic recovery after chemotherapy was normal. It is therefore expected that the radio protection/chemo protection of normal gut is safe as long as LIF treatment and delivery is both local and transient, and there is no tumor locally in the gut. A phase I study of recombinant LIF human leukemia inhibitory factor in patients with advanced cancer. (60) It is known that LIF has an inhibitory effect on leukemia, therefore administering therapeutically or prophylactically effective amounts of LIF to treat or prevent GVHD in patients with leukemia who will have or have had bone marrow transplants is expected to be safe. (Gearing D., et al., Molecular cloning and expression of cDNA encoding a murine myeloid leukemia inhibitory factor (LIF). EMBO J., 1987; 6:3995-4002.

LIF Variants and Fragments

LIF pertains to a natural LIF sequence or active fragments or active variants thereof. A variant of LIF may be a naturally occurring variant, for example a variant which is expressed by a non-human species. Also included as variants of LIF are sequences which vary from SEQ ID NO: 1 but are not necessarily naturally occurring. Over the entire length of the amino acid sequence of SEQ ID NO: 1, a variant will preferably be at least 90% homologous to that sequence based on amino acid identity. More preferably, the variant is at least 95%, 97% or 99% homologous based on amino acid identity to the amino acid sequence of SEQ ID NO: 1 or SEQ ID NO. 2 (amino acids 50-160 of human LIF) over the entire sequence. Homology may be determined using any method known in the art. For example, the UWGCG Package provides the BESTFIT program which can be used to calculate homology, for example used on its default settings (Devereux et al (1984) Nucleic Acids Research 12, p 387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10.

It is expected that fragments of LIF that contain a region of the cytokine that binds to the target LIF receptor (LIFR) will have the same or nearly the same biological activity that LIF has to increase ISC regeneration, that in turn increases IEC generation. The interaction between human LIF and human LIFR was studied by Hudson, et al. using a set of site mutation constructs, and Huyton et al, analyzed the crystal structure of human LIF protein bound to a portion of mouse LIFR. (58, 59) An unusual cytokine:Ig-domain interaction was revealed in the crystal structure of leukemia inhibitory factor (LIF) in complex with the LIF receptor. Collectively, these studies showed that the regions of LIF from aa51-aa59; aa103-aa-107 and aa153-aa160 are involved in the interaction of LIF with the receptor. Among these, aa 51, 151, 156 and 159 are critical for binding; contact between ligand and receptor is important to form a stable structure. Huyton T., et al. showed that “LIF first hinds to the LIF receptor (LIFR) with low nanomolar affinity and then to gp130 to form a high-affinity (picomolar) functional signaling complex (2-4).” The LIF binding to LIFR is sufficient to trigger the further interaction of the LIF:LIFR:gp130 and downstream biological activity. The complex contains LIF:LIFR/gp130. According to the structural studies, LIF binds to LIF-R but not directly to gp130. For certain embodiments of the present invention, a biologically active fragment of LIF for in vivo therapeutic use or in vitro use comprises at least aa51-aa59; aa103-aa-107 and aa153-aa160, in a preferred embodiment the biologically active fragment is SEQ ID NO:2 (aa 51-160.) or an active fragment that includes aa 51-160. Other fragments for use in the methods and products of the invention include fragments that additionally comprise amino acids before aa 51 and after 160, such as for example aa 40-180. Biologically active fragments of LIF for use in the present inventions can be screened using routine methods, such as in vitro Immunoprecipitation (IP) pull down (62), which is the simplest method, and in vivo biological testing to see if the fragment can activate the LIF downstream signaling can also be used.

### Discussion

The results from the studies using a mouse model system show that the expression of LIF in the stem cell niche enables ISCs and progenitors to proliferate and maintain homeostasis of the intestinal epithelial tissue acting by increasing β-catenin activity mainly through the AKT/GSK3. Compared with WT mice, intestinal crypts of LIF KO mice are smaller and contain less ISCs, Paneth cells and proliferating TA cells. Further, viable LIF KO mice show a retarded postnatal growth rate. LIF is not only essential to maintain homeostasis of the intestinal epithelium, but also important for the regeneration of epithelium in response to injury. The intestinal epithelial regeneration induced by IR is severely compromised in LIF KO mice.

LIF KO mice have a reduced lifespan after 12-Gy whole body IR compared with WT mice. Administering recombinant mouse LIF can rescued the impaired intestinal epithelial regeneration in response to whole body IR and restore the lifespan of LIF KO mice to a similar extent as WT mice. More importantly, LIF promoted the intestinal epithelial regeneration of WT mice after IR and prolonged their lifespans. Unexpectedly, LIF acted to reduce radiation injury, and also GvHD, by increasing β-catenin activity mainly through the AKT/GSK3 and not through STAT4 and STAT3/YAP signaling as was reported in the case of experimental colitis.

Results demonstrate that LIF regulates the function of ISCs by upregulating β-catenin activity mainly through the AKT/GSK3β signaling. β-catenin is a critical regulator of adult stem cells, including ISCs. β-catenin fulfills its stem cell-associated functions through its nuclear translocation to interact with TCF/LEF, which leads to the induction of β-catenin target genes, whose products play essential roles in stem cell self-renewal and homeostasis of epithelial tissues (15, 16). Loss of LIF significantly reduced nuclear localization of β-catenin and decreases the expression of β-catenin target genes. Further, we found that LIF up-regulated β-catenin function through the AKT/GSK3β signaling. AKT activates β-catenin through inactivation of GSK3β, a negative regulator of β-catenin. LIF up-regulated AKT activity in the intestine; loss of LIF decreases AKT activity in the intestine in the LIF KO mice, which in turn increases GSK3β activity, leading to the decreased β-catenin activity. While ISCs of WT mice are sensitive to AKT inhibition by Wortmannin and Capivasertib, AKT inhibition has a much less pronounced effect on ISCs of LIF KO mice. Activating AKT by SC79 greatly rescues the function of ISCs of LIF KO mice to a similar extent as supplementation with recombinant mouse LIF protein. B locking GSK3β activity by CHIR99021 largely rescued the impaired function of ISCs of LIF KO mice. These results indicate that LIF activates the β-catenin signaling mainly through its regulation of the AKT/GSK3β signaling to ensure intestinal epithelial tissue homeostasis under physiological conditions and upon injury such as IR or in cases of GvHD.

Pharmaceutical Formulations and Administration

The pharmaceutical compositions/formulations discussed herein comprising an enumerated agent (LIF, biologically active fragments of LIF and variants thereof) are suitable for treating a subject who will receive or has received an autologous or noon-autologous transplant, such as a bone marrow transplant and GI diseases and disorders in mammals, preferably human subjects, using the present methods. The results herein show that LIF and the other active agents are effective in treating radiation injury and GvHD. Other diseases that can be treated by the present methods and compositions include inflammatory bowel disease, Crohn's disease, colitis including an autoimmune response or an inflammatory response that results in damage to the intestinal epithelium. The enumerated diseases further include all diseases or disorders that pertain to damage to ISCs or the intestinal epithelium. This includes diseases of the first, second, and third part of the duodenum, jejunum, ileum, the ileo-cecal complex, large intestine (ascending, transverse, and descending colon), sigmoid colon, rectum, and anus.

Emfilermin is the term used for rhLIF produced in Escherichia coli. Administration of a single s.c. dose in healthy volunteers found that rhLIF was safe and well tolerated up to doses of 4 μg/kg. (60). In preferred embodiments, the active agents including LIF, biologically active fragments and variants are preferably human, and are typically recombinant peptides.

The effective amount of an enumerated (active) agent and variants thereof used therapeutically in a subject in the in vivo methods described herein will vary. In general, an effective amount is between about 0.5 μg/kg/day and 100 μg/kg/day, and includes between about 0.5 μg/kg and 100 μg/kg, between about 1 μg/kg and 75 μg/kg, between about 5 μg/kg and 50 μg/kg, between about 20 μg/kg and 40 μg/kg and includes about 0.5 μg/kg, 1 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, and 60 μg/kg. Methods for diagnosis of and monitoring the extent of GI diseases including RI and GvHD are routine in the art and it is standard practice to evaluate the response of a subject being treated for an enumerated disease to determine efficacy of treatment, including dosage and frequency of administration.

In addition to continuous administration using osmotic pumps, active agents can be administered as a single treatment or, preferably, can include a series of treatments, that continue at a frequency and for a duration of time that causes one or more symptoms of the enumerated disease to be reduced or ameliorated, or that achieves the desired effect including effects of increasing insulin secretion and serum insulin, increasing insulin sensitivity, increasing glucose tolerance, decreasing weight gain, decreasing fat mass, and causing weight loss.

It is understood that the appropriate dose of an active agent depends upon a number of factors within the ken of the ordinarily skilled physician, veterinarian, or researcher. The dose(s) vary, for example, depending upon the identity, size, and condition of the subject or sample being treated, further depending upon the route by which the composition is to be administered, and the effect which the practitioner desires the an active agent to have. It is furthermore understood that appropriate doses of an active agent depend upon the potency with respect to the activity to be modulated. Such appropriate doses may be determined for treatment of a GI disease or disorder or for GvHD by determining the response of the subject to treatment regarding intestinal health using routine methods, including if necessary via a biopsy containing ISC and IEC. When an active agent is administered to an animal (e.g., a human) in order to treat a GI disease including radiation injury or GvHD, a relatively low dose may be prescribed at first, with the dose subsequently increased until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Therefore, the forms which the pharmaceutical composition can take will include, but are not limited to: tablets, capsules, caplets, lozenges, dragees, pills, granules, oral solutions, powders for dilution, powders for inhalation, sterile solutions or other liquids for injection or infusion, transdermal patches, buccal patches, inserts and implants, rectal suppositories, oils, ointments, suspensions, emulsions, lipid vesicles, and the like.

Treatment regimens include a single administration or a course of administrations lasting two or more days, including a week, two weeks, several weeks, a month, two months, several months, a year, or more, including administration for the remainder of the subject's life. The regimen can include multiple doses per day, one dose per day or per week, for example, or a long infusion administration lasting for an hour, multiple hours, a full day, or longer. In certain embodiments, administration of the active agent is begun prior to radiation treatment that is given to treat cancer and is continued during and sometimes after the radiation treatment is completed. for reducing or ameliorating symptoms of RI and GvHD, for example, administration would begin prior to IR or transplant and similarly continue afterward for as long as a physician would determine is desirable in some cases, treatment could be long term, even years.

The pharmaceutical compounds discussed herein can be present in the form of pharmaceutically acceptable salts, acids, hydrates, and solvates, or as a base. These compounds can exist in amorphous form or in any crystalline form. Preferably, the pharmaceutical compositions comprise a therapeutically effective amount. Extended and sustained release compositions also are contemplated for use with and in the inventive embodiments as described in detail below. Suitable carriers include any of the known ingredients to achieve a delayed release, extended release or sustained release of the active components. The pharmaceutical compositions of the invention include the base, and any pharmaceutically acceptable hydrate, solvate, acid or salt, and can be amorphous or in any crystalline form, or as an oil or wax. Any pharmaceutically acceptable salt can be used, as may be convenient. Generally, these salts are derived from pharmaceutically and biologically acceptable inorganic or organic acids and bases or metals. Examples of such salts include, but are not limited to: acetate, adipate, alginate, ammonium, aspartate, benzoate, benzenesulfonate (besylate), bicarbonate, bisulfate, butyrate, citrate, camphorate, camphorsulfonate, carbonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, magnesium, maleate, malonate, methanesulfonate (mesylate), 2-naphthalenesulfonate, nicotinate, nitrate, oxalate, palmoate, pectinate, persulfate, 3-phenylpropionate, phosphate, picrate, pivalate, potassium, propionate, salicylate, sodium, succinate, sulfate, tartrate, thiocyanate, toluenesulfonate (tosylate) and undecanoate salts.

The compounds also include any or all stereochemical forms of the therapeutic agents (i.e., the R and/or S configurations for each asymmetric center). Therefore, single enantiomers, racemic mixtures, and diastereomers of the therapeutic agents are within the scope of the invention. Also within the scope of the invention are steric isomers and positional isomers of the therapeutic agents. The therapeutic agents of some embodiments are also meant to include compounds which differ only in the presence of one or more isotopically enriched atoms. For example, therapeutic agents in which one or more atom is replaced by, for example, deuterium, tritium, 13C, 14C (or any isotopic labels as commonly used in the art such as phosphorus, calcium, iodine, chlorine, bromine, or any other convenient element for isotopic labeling) are within the scope of this invention.

Enteral formulations In preferred embodiments, the enumerated compounds (LIF, LIF biologically active fragments and variants) described herein are formulated for enteral administration or administration locally to the intestine including the colon, and are administered as a pharmaceutical composition that includes a pharmaceutically acceptable carrier. The terms “pharmaceutically acceptable carrier,” “pharmaceutically acceptable excipient,” or “pharmaceutically acceptable vehicle” refer to any convenient compound or group of compounds that is not toxic and that does not destroy or significantly diminish the pharmacological activity of the therapeutic agent with which it is formulated. Such pharmaceutically acceptable carriers or vehicles encompass any of the standard pharmaceutically accepted solid, liquid, or gaseous carriers known in the art, such as those discussed in the art. Such pharmaceutically acceptable carriers or vehicles encompass any of the standard pharmaceutically accepted solid, liquid, or gaseous carriers known in the art, such as those discussed in the art. The formulations can also include sterile diluents, water, saline solution, fixed oils, polyethylene glycols, glycerine, propylene glycol, antibacterial agents (e.g., benzyl alcohol, methyl parabens, and the like), antioxidants (e.g., ascorbic acid, sodium bisulfite, and the like), chelating agents (e.g., EDTA and the like), buffers (e.g., acetate, citrate, phosphate, carbonate and the like), tonicity-adjusting compounds (e.g., sodium chloride, dextrose, polyalcohols, and the like), and pH adjusters, (e.g., acids or bases).

A suitable carrier depends on the route of administration contemplated for the pharmaceutical composition. Routes of administration are determined by the person of skill according to convenience, the health and condition of the subject to be treated, and the location and stage of the condition to be treated. Such routes can be any route which the practitioner deems to be most effective or convenient using considerations such as the patient, the patient's general condition, and the specific condition to be treated. Routes of administration include, but are not limited to: enteric, local or parenteral, oral, intravenous, intraarterial, intrathecal, subcutaneous, intradermal, parenteral, intraperitoneal, rectal, topical, nasal, local injection, buccal, transdermal, sublingual, transmucosal, and the like.

Administration of an enumerated agent can be by transfusion or infusion, and can be administered by an implant, an implanted pump, an external pump, or any device known in the art to facilitate delivery directly or indirectly to the small and large intestines. In an embodiment the composition is delivered primarily to the colon via a colon-specific drug delivery method. Possible intestine- and colon-specific drug delivery methods include, but are not limited to, the following: pH-dependent release, microbially-triggered drug delivery, conjugates, time-controlled delivery, osmotically-regulated delivery, pressure-controlled delivery, multi matrix delivery systems, bioadhesion, microparticulate or nanoparticulate delivery. The drug delivery method may include any combination of intestine- and colon-specific drug delivery methods. For example, a compound may be formulated to minimize release of the drug in the stomach, small intestine, and/or upper GI tract and to promote release of the drug in the colon. Similarly formulations can target the small intestine and not the colon, or a particular region of the small intestine. In one example, the route of administration may be topical, enteral, parenteral or a combination thereof.

Enteral administration routes include non-local administration of an enumerated agent via the digestive tract. Common enteral routes include, but are not limited to: oral, rectal, sublingual, sublabial, buccalor a combination thereof. The mechanism for drug absorption from the intestine is for most drugs passive transfer. Topical routes include local administration of an enumerated agent directly onto the affected area in the intestinal tract, where the agent diffuses through the lipid cell membrane of the epithelial cells lining the inside of the intestines. The rate at which the agent diffuses is largely determined by ionization and lipid solubility.

Drugs are normally well absorbed from the gastrointestinal (G.I.) tract and dosage forms such as capsules, tablets, and suspensions are well accepted by the general population. Drug delivery systems targeted to the intestines including the colon are known in the art to include covalent linkage compositions, polymer coated compositions, compositions embedded in matrices, time release compositions, redox-sensitive polymer compositions, bioadhesive compositions, microparticle coating compositions, and osmotic delivery compositions. See U.S. Pat. No. 8,470,885. A number of different formulations are available for delivery of desired compositions comprising the enumerated agents to the intestines including amylose coated tablets, enterically coated chitosan tablets, matrix within matrix or multimatrix systems or polysaccharide coated tablets. Multimatrix controlled release systems are disclosed in U.S. Pat. No. 7,421,943. Therefore in some embodiments the pharmaceutical compositions are administered orally or locally to the colon or in formulations that target them for absorption in the intestines, including in a specific region of the intestine such as the jejunum, ileum or duodenum or it can be administered by implanting an osmotic pump, for example at a site or subcutaneous location that is proximal to the targeted region of the intestine.

One form of enteral administration is via an enteric capsule for drug delivery (ECDD) is suitable for the present embodiments, in which oral delivery with full enteric protection and rapid release in the upper gastrointestinal (GI) tract is achieved without the use of coatings. ECDD's intrinsically enteric properties are attained by incorporating pharmaceutically approved enteric polymers in the capsule shell using conventional pin-dipping capsule manufacturing processes. ECDD enables the oral delivery of sensitive molecules, such as nucleotides and peptides. The enteric properties and rapid release of specialized ECDD capsule shells have been demonstrated to meet pharmacopeia standards for both in vitro and in vivo performance using esomeprazole magnesium trihydrate (EMT) as a model compound. Hassan Benameur, Technical Brief 2015 Volume 1, Particle sciences, drug development services, page 34-37. Capsule Drug Delivery Technology—Achieving Protection Without Coating.

Controlled release dosage forms that will provide therapy over an extended period of time come within the scope of the invention. Normally this would be once a day and it is believed that such a change in dosage regimen will reduce adverse reactions and side effects and also improve patient compliance. The design and evaluation of controlled release dosage forms must, however, take into account the properties of the G.I. tract, including the rapid transit of material through the small intestine. The use of synthetic polymers that may have muco- or bio-adhesive properties has been investigated and is disclosed in WO 85/02092.

The term “slow release” refers to the release of a drug from a polymeric drug delivery system over a period of time that is typically more than one day, wherein the active agent is formulated in a polymeric drug delivery system that releases effective concentrations of the drug. Drug delivery systems may include a plurality of polymer particles containing active drug material, each of the particles preferably having a size of 20 microns or less, and incorporating on the outer surface of at least some of the particles a bioadhesive material derived from a bacterium such that in use the bioadhesive material will adhere to the small intestine of the gut. Such drug delivery systems have been described in U.S. Pat. No. 6,355,276. The use of these microorganisms in the design allow for a controlled release dosage form with extended gastrointestinal residence.

In some embodiments a slow release preparation comprising the active agents is formulated. It is desirable to prolong delivery with these slow release preparations so that the drug may be released at a desired rate over this prolonged period. By extending the period, the drug can if required be released more slowly, which may lead to less severe adverse reactions and side effects. The preparation of sustained, controlled, delayed or anyhow modified release form can be carried out according to different known techniques: 1. The use of inert matrices, in which the main component of the matrix structure opposes some resistance to the penetration of the solvent due to the poor affinity towards aqueous fluids; such property being known as lipophilia; 2. The use of hydrophilic matrices, in which the main component of the matrix structure opposes high resistance to the progress of the solvent, in that the presence of strongly hydrophilic groups in its chain, mainly branched, remarkably increases viscosity inside the hydrated layer; and 3. The use of bioerodible matrices, which are capable of being degraded by the enzymes of some biological compartment. See. U.S. Pat. No. 7,431,943.

In certain embodiments, dosage forms of the compositions of the present invention include, but are not limited to, implantable depot systems. In one embodiment. the depot system includes the active agent embedded in a three-dimensional matrix. The three-dimensional matrices to be used are structural matrices that provide a scaffold to hold and support the cells, and are porous to allow fluid flow. Scaffolds can take forms ranging from fibers, gels, fabrics, sponge-like sheets, and complex 3-D structures with pores and channels fabricated using complex Solid Free Form Fabrication (SFFF) approaches. As used herein, the term “scaffold” means a three-dimensional (3D) structure (substrate and/or matrix). It may be composed of biological components, synthetic components or a combination of both. Further, it may be naturally constructed by cells or artificially constructed. In addition, the scaffold may contain components that have biological activity under appropriate conditions. The structure of the scaffold can include a mesh, a sponge or can be formed from a hydrogel. In certain embodiments, the scaffold is biodegradable.

Examples of biodegradable depot systems include but are not limited to PLGA based injectable depot systems; non-PLGA based injectable depot systems, and injectable biodegradable gels or dispersions. Each possibility represents a separate embodiment of the invention. The term “biodegradable” as used herein refers to a component which erodes or degrades at its surfaces over time due, at least in part, to contact with substances found in the surrounding tissue fluids, or by cellular action. In particular, the biodegradable component is a polymer such as, but not limited to, lactic acid-based polymers such as polylactides e.g. poly (D,L-lactide) i.e. PLA; glycolic acid-based polymers such as polyglycolides (PGA) e.g. Lactel® from Durect; poly (D,L-lactide-co-glycolide) i.e. PLGA, (Resomer® RG-504, Resomer® RG-502, Resomer® RG-504H, Resomer® RG-502H, Resomer® RG-504S, Resomer® RG-502S, from Boehringer, Lactel® from Durect); polycaprolactones such as Poly(e-caprolactone) i.e. PCL (Lactel® from Durect); polyanhydrides; poly(sebacic acid) SA; poly(ricenolic acid) RA; poly(fumaric acid), FA; poly(fatty acid dimmer), FAD; poly(terephthalic acid), TA; poly(isophthalic acid), IPA; poly(p-{carboxyphenoxy} methane), CPM; poly(p-{carboxyphenoxy} propane), CPP; poly(p-{carboxyphenoxy}hexane)s CPH; polyamines, polyurethanes, polyesteramides, polyorthoesters {CHDM: cis/trans-cyclohexyl dimethanol, HD:1,6-hexanediol. DETOU: (3,9-diethylidene-2,4,8,10-tetraoxaspiro undecane)}; polydioxanones; polyhydroxybutyrates; polyalkylene oxalates; polyamides; polyesteramides; polyurethanes; polyacetals; polyketals; polycarbonates; polyorthocarbonates; polysiloxanes; polyphosphazenes; succinates; hyaluronic acid; poly(malic acid); poly(amino acids); polyhydroxy valerates; polyalkylene succinates; polyvinylpyrrolidone; polystyrene; synthetic cellulose esters; polyacrylic acids; polybutyric acid; triblock copolymers (PLGA-PEG-PLGA), triblock copolymers (PEG-PLGA-PEG), poly (N-isopropylacrylamide) (PNIPAAm), poly (ethylene oxide)-poly (propylene oxide)-poly (ethylene oxide) triblock copolymers (PEO-PPO-PEO), poly valeric acid; polyethylene glycol; polyhydroxyalkylcellulose; chitin; chitosan; polyorthoesters and copolymers, terpolymers; lipids such as cholesterol, lecithin; poly(glutamic acid-co-ethyl glutamate) and the like, or mixtures thereof.

Self emulsifying microemulsion drug delivery systems (SMEDDS) are known in the art as effective delivery systems into the G.I. tract. See U.S. Patent Application 2001/00273803. The term SMEDDS is defined as isotropic mixtures of oil, surfactant, cosurfactant and drug that rapidly form oil in water microemulsion when exposed to aqueous media or gastrointestinal fluid under conditions of gentle agitation or digestive motility that would be encountered in the G.I. tract.

Thermostable nanoparticles may be contained in a drug delivery system targeted for the G.I. tract. See U.S. Patent Application 2000/60193787. These drug delivery systems may include at least one type of biodegradable and/or bioresorbable nanoparticle and at least one drug that possesses at least one of the following properties: emulsifier or mucoadhesion. The drug may substantially cover the surface of the nanoparticle and may be used for delivering at least one drug across a mucosal membrane such as the lining of the gut. Certain medications, for example resins that prevent bile acid absorption, can be used in the pharmaceutical formulations of the present invention.

Enumerated agents may be coated, in some embodiments, with a pH-dependent polymer that inhibits or minimizes release of the drug in the stomach, small intestine, and/or upper GI tract and promotes release of the drug in the colon. The drug core may include tablets, capsules, pellets, granules, microparticles, nanoparticles, or a combination thereof. A suitable pH-dependent polymer may be a derivative of acrylic acid or cellulose. Examples of suitable pH-dependent polymers include, but are not limited to: hydroxypropyl methylcellulose phthalate, hydroxypropyl methylcellulose acetate succinate, acrylic polymers, acrylic copolymers, methyl methacrylate-methacrylic acid copolymers, cellulose acetate phthalate, cellulose acetate succinate, cellulose acetate trimellate, Eudragit®L100, Eudragit®L100-55, Eudragit®5100, Eudragit®L-30D, Eudragit®FS 30D, hydroxypropyl methylcellulose phthalate 50, hydroxypropyl methylcellulose phthalate 55, or any combination thereof.

The active agent may be embedded in a polymer matrix, wherein the polymer is pH-dependent. The drug delivery system may comprise Eduracol®technology, wherein a drug core is enclosed by multiple layers of Eudragit®. The drug delivery may comprise enclosure of the active agent by multiple layers of pH-dependent polymers. The timing of the drug release may be controlled by the thickness and composition of the multiple layers surrounding the drug core.

The active agent may be formulated to comprise a biodegradable polymer that reacts with the microflora or enzymes present in the colon, thereby facilitating delivery of the agent to the colon and inhibiting release of the drug in the stomach or small intestine. The active agent may be coated with a biodegradable polymer. The active agent may be embedded in biodegradable polymeric matrices or hydrogels. The drug delivery system may comprise both biodegradable polymers and pH-dependent polymers. The biodegradable polymer may be selected to react with a targeted colonic bacteria or enzyme. Examples of enzymes that a biodegradable polymer may react with include, but are not limited to: azoreductase, bacterial hydrolase glycosidase, esterase, polysaccharidase, and the like. For example, biodegradable polymers containing azoaromatic linkages may react with azoreductase. Examples of suitable biodegradable polymers include, but are not limited to: azo polymers, polyurethane containing azo aromatic groups, azo polymers using styrene-2-hydroxyethyl methacrylate and divinyl azobenzene as a cross linker, copolymers of 2-hydroxyethyl methacrylate with bis(methacryloylamino)azo benzene as a cross linker, terapolymers of methyl methacrylate, 2-hydroxyethyl methacrylate and methacrylic acid with N,N-bis[(methacryloyloxyethyl)oxy(carbonylamino)]azo benzene, divinyl azobenzene, and bis(methacryloylamino)azobenzene as a cross linker, poly(ether-ester) azopolymers, urethanes containing azo aromatic linkages in the backbone, polygalactomannans in polymethacrylate solutions, inulin and copolymers of acrylic acid esters, pectin and ethyl cellulose, glutaraldehyde cross-linked dextran, poly(-caprolactone), polylactic acid, poly(lactic-co-glycolic acid), polysaccharides, amylose, guar gum, pectin, chitosan, inulin, cyclodextrin, chondroitin sulphate, dextran, locust bean gum, arabinogalactan, chondroitin sulfate, xylan, calcium pectinate, pectin/chitosan mixtures, amidated pectin, or any combination thereof. Other examples and teachings of targeted delivery in the gut of a subject are provided in US Pat. Pub. 20160303133.

### EXAMPLES

### Example 1 Materials and Methods (IR) Treatment

LIF KO mice established by Dr. Stewart (PMID: 1522892, 19) in C57BL/6J background were obtained from EMMA repository (European Mouse Mutant Archive; EM:02619). Lgr5-EGFP-IRES-creERT2 mice (Lgr5-GFP; stock #008875, Jackson Laboratory were purchased from the Jax Laboratory. LIF KO mice were bred with Lgr5-GFP mice to produce LIF KO; Lgr5-GFP mice. Eight-12 week-old LIF KO and WT littermates were used for experiments.

For IR treatment, age- and gender-matched LIF KO and WT mice at 8-12-week-old were subjected to 9-16 Gy whole body IR with a 137 Cs γ-source irradiator. Mice were sacrificed 72 h after IR to collect tissues for further experiments. For LIF treatment, mice were injected with recombinant mouse LIF (Millipore) (i.p., 30 ng/g body weight twice a day for 7 days, starting from 3 days before IR). All animal experiments were performed with the approval of the Institutional animal Care and Use Committee of Rutgers State University of New Jersey. This amount of radiation (12 Gy whole body IR) was expected to be lethal. 12-13 Gy whole body IR was used in the initial experiments because this condition will lead to a GI syndrome and is suitable as a model to study the response of intestinal epithelium towards radiation injury as well as any intestinal epithelial damage. The cause of death in mice treated with 12 Gy whole body IR is due to the GI syndrome.

### Crypt Isolation and Organoid Culture

Crypt isolation and organoid culture were performed as described previously (Nan Gao's paper, 2015, Development). Crypts were isolated from the mouse small intestine. 150-200 crypts were embedded in growth factor-reduced Matrigel and cultured with mouse ENR organoid culture medium containing 1×B27 (Life Tech Gibco), 1×N2 (Life Tech Gibco), 1 mM N-acetylcysteine (Sigma), 50 ng/ml of EGF (Life Technology), 100 ng/ml of Noggin (R&D System), and 1 μg/ml of R-spondin 1 (R&D system). Recombinant mouse LIF (Millipore), wortmannin (Cell Signaling Technology) and CHIR99021 (Stemgent) were used for organoid treatment.

### Organoid Measurement

The size organoid was evaluated by quantify surface area of horizontal cross section of organoids acquired from multiple random non-overlapping pictures by an Olympus inverted microscope using ImageJ software. The vertical direction of expansion was not included in the calculations. n≥30/group. The percentages of budding organoids were analyzed by light microscopy at different days during organoid culture. n≥100/group.

### Histology

Paraffin-embedded small intestine tissues were sectioned with 5 μm thickness and stained with hematoxylin and eosin (H&E) as described previously (Zhao Y, 2018, eLife).

### IHC Staining Assays

Di-identified normal human colorectal tissue samples were obtained from Princeton Cancer Tissue Repository with an IRB approval. IHC staining was performed as previously described (PMID:22509031). Anti-LIF (Novus, NBP2-27406, 1:500 dilution) antibodies were used to detect the levels of LIF in mouse and human tissues. Anti-lysozyme (Abcam, ab108508, 1:4000 dilution), anti-Olfm4 (Cell signaling, cat #39141, 1:1000 dilution), anti-β-catenin (BD Biosciences, cat #610153, 1:500 dilution), anti-CD44 (BioLegend, 156002, 1:500 dilution) and anti-Ki67 (Abcam, ab16667, 1:200 dilution) antibodies were used to detect the levels of lysozyme, Olfm4, β-catenin, CD44 and Ki67 in mouse tissues.

### IF Staining Assays

IF staining of organoids was performed as previously described (Gao and Kaestner, 2010). In brief, organoids were fixed with 4% paraformaldehyde for 10 min, treated with 0.5% TritonX-100 for 10 min and blocked with 10% goat serum in IF buffer (0.1% BSA, 0.2% Triton X100, 0.05% Tween-20, 0.05% NaN₃ in PBS) for 2 hours. Organoids were incubated with anti-Ki67 (Abcam, ab16667, 1:200 dilution) and anti-GFP (Abcam, ab6673, 1:1000 dilution) antibodies overnight at room temperature and then incubated with Alexa Fluor® 555 Goat Anti-Rabbit IgG (H+L) (Invitrogen, 1:800 dilution) and Alexa Fluor® 488 Donkey Anti-Goat IgG (H+L) (Invitrogen, 1:200 dilution), respectively to detect Ki67 and Lgr5-EGFP. Nuclei were stained with 4′, 6-diamidino-2-phenylindole (DAPI; Vector).

Immunofluorescent staining of mouse intestine tissues was performed as previously described (Gao, N. & Kaestner, K. H. Cdx2 regulates endo-lysosomal function and epithelial cell polarity. Genes Dev 24, 1295-1305 (2010)). In brief, tissue sections were deparaffinized in xylene and rehydrated with ethanol. After pre-incubation with 2% BSA and 2% normal goat serum in PBST for 1 hour, tissue sections were incubated with anti-E-Cadherin (BD, 610181, 1:50 dilution), anti-LIF (Novus, NBP2-27406, 1:500 dilution), anti-lysozyme (Abcam, ab108508, 1:250 dilution), anti-Olfm4 (Cell signaling, 39141, 1:500 dilution) and anti-GFP (Abcam, ab6673, 1:1000 dilution) antibodies overnight at 4° C. Slides were then incubated with Alexa Fluor® 555 Goat Anti-Rabbit IgG (H+L) or Alexa Fluor® 488 Goat Anti-mouse IgG (H+L) (Invitrogen, 1:800 dilution). Nuclei were stained with DAPI. Images were captured using a confocal microscope (Nikon A1R Si).

### Western-Blot Assays

Standard Western-blot assays were used to analyze protein expression in small intestine tissue. The following antibodies were used for assays: antibodies against AKT (Santa Cruz, SC-1618, 1:2000 dilution), p-AKT (Phospho Ser473) (Cell Signaling Technology, 9271, 1:2000 dilution), anti-GSK3β (Cell signaling, 9315; 1:1000 dilution, anti-GSK3β (Phospho Ser9) (Abcam, ab131097; 1:1000 dilution) and anti-β-actin (A5441, Sigma; 1:125,000 dilution) antibodies.

### Quantitative Real-Time PCR

Total RNA from mice small intestine tissues or organoids was prepared by using an RNeasy kit (Qiagen). Quantitative real-time PCR assays were performed as described previously. The sequence of primers for real-time PCR was listed in Table 1. The expression of genes was normalized with the β-actin gene. Data was obtained from 5 independent biological samples with 3 technical replicates.

### Single Cell Data Analysis

Single cell data analysis was performed by analyzing a public available dataset of small intestinal epithelium single-cell RNA (GSE92332-AtlasFullLength-TPM).

### Statistical Analysis

The data were expressed as mean±SD. All p-values were obtained using two-tailed Student t-tests or Fisher's Exact Test. Values of p<0.05 were considered to be significant.

### Source of Recombinant LIF Protein

LF, recombinant mouse (Millipore, Sigma Cata #: LIF2050)

TABLE 1
The sequences of the primers for real-time PCR

| Gene | Primer |
|---|---|
| Olfm4 | Forward: 5′ GCCACTTTCCAATTTCAC 3′ |
| | SEQ ID NO: 3 |
| | Reverse: 5′ GAGCCTCTTCTCATACAC 3′ |
| | SEQ ID NO: 4 |
| Lyz | Forward: 5′ GGTGGTGAGAGATCCCCAAG 3′ |
| | SEQ ID NO: 5 |
| | Reverse: 5′ CAGACTCCGCAGTTCCGAAT 3′ |
| | SEQ ID NO: 6 |
| Axin 2 | Forward: 5′ TGAGATCCACGGAAACAGC 3′ |
| | SEQ ID NO: 7 |
| | Reverse: 5′ GTGGCTGGTGCAAAGACAT 3′ |
| | SEQ ID NO: 8 |
| Ascl 2 | Forward: 5′ TCCAGTTGGTTAGGGGGCTA 3′ |
| | SEQ ID NO: 9 |
| | Reverse: 5′ GCATAGGCCCAGGTTTCTTG 3′ |
| | SEQ ID NO: 10 |
| Lgr5 | Forward: 5′ CCTACTCGAAGACTTACCCAGT 3′ |
| | SEQ ID NO: 11 |
| | Reverse: 5′ GCATTGGGGTGAATGATAGCA 3′ |
| | SEQ ID NO: 12 |
| Wnt3 | Forward: 5′ CTTCTAATGGAGCCCCACCT 3′ |
| | SEQ ID NO: 13 |
| | Reverse: 5′ GAGGCCAGAGATGTGTACTGC 3′ |
| | SEQ ID NO: 14 |
| β-Actin | Forward: 5′ GAACCCTAAGGCCAACCGTGAAAAGATG |
| | AC 3′ SEQ ID NO:15 |
| | Reverse: 5′ GCAGGATGGCGTGAGGGAGAGCA 3′ |
| | SEQ ID NO: 16 |

The Justification of Using i.p. Route in Mouse Experiments

It has been shown that “intravenous injection of 2 μg of LIF resulted in a very short serum half-life with a second phase of 8 to 9 minutes (data not shown). We chose i.p. route to get a more sustained elevation of serum LIF levels to ensure the effect of LIF in the intestine. Any adverse effects will not be that fast. However, the i.p. injection of 2 μg LIF resulted in a sustained elevation of serum LIF levels that exceeded 1,000 U/mL for approximately 3 hours.” “The very short serum half-life of intravenously injected LIF indicated that the i.p. route was more practicable in mice for ensuring sustained periods of elevated serum LIF levels.” (65) Therefore, i.p. route was chosen in the above-mentioned experiments.

The range of irradiation dosage used in mouse model system studying intestinal injury induced by irradiation were between 8-16 Gy. (39, 63, 64).

### Example 2 Intestinal Epithelial Cells in the Crypt Express LIF

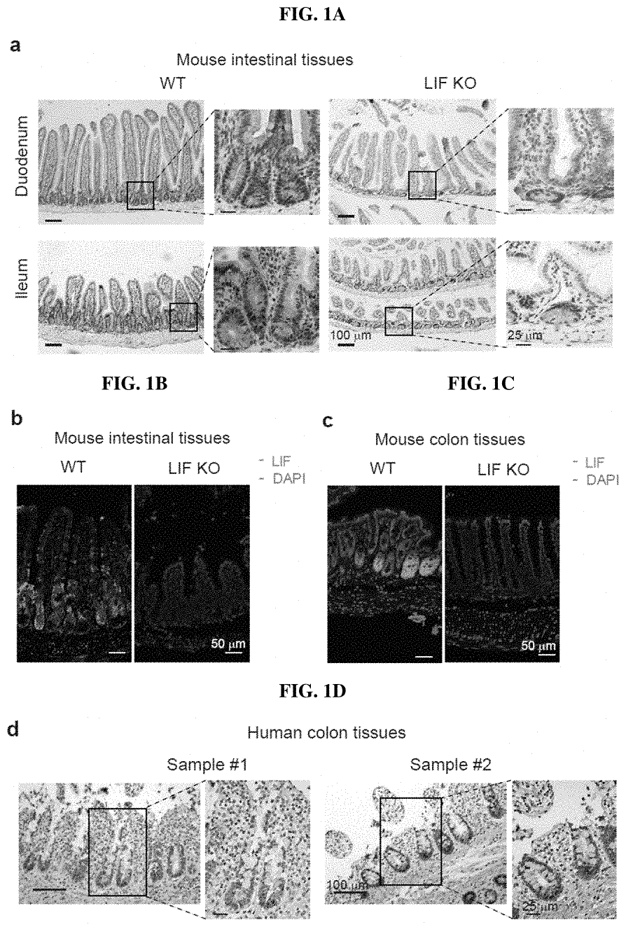

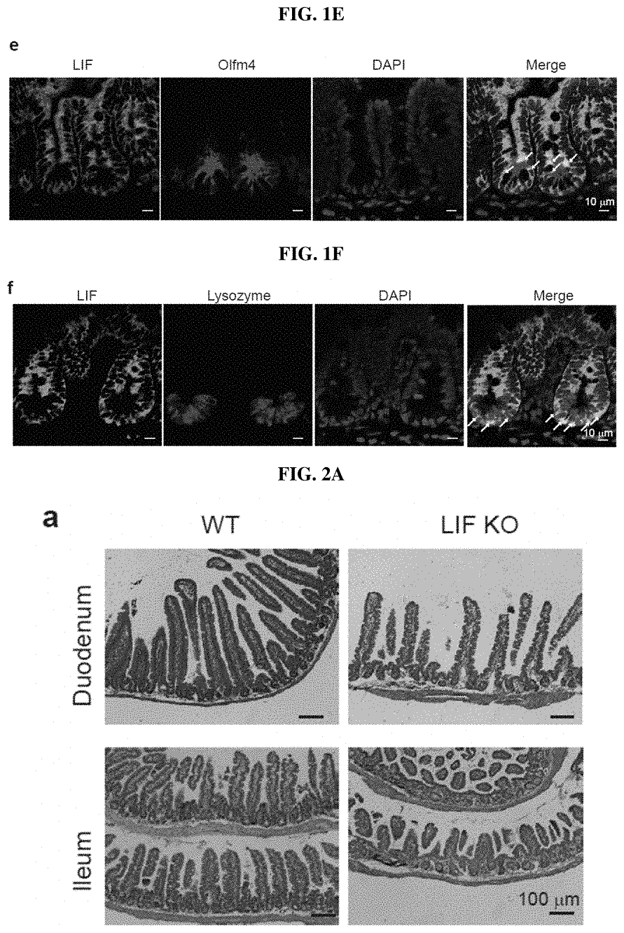

Immunohistochemistry (IHC) staining and immunofluorescence (IF) staining using an LIF antibody showed the expression pattern of LIF in the mouse intestinal tissue, specifically in the epithelium along the whole intestine including duodenum, ileum and colon (FIG. 1A-1C). Notably, the majority of the cells with positive LIF staining we localized in the crypt in WT mice (FIG. 1A-1C). Importantly, LIF expression was also observed in the epithelial cells in the crypt of normal human colon tissues (FIG. 1D).

The intestinal crypts contain different types of cells, including ISCs, Paneth cells and TA cells. Using Olfm4 and Lysozyme, markers of ISCs and Paneth cells, respectively, for co-staining, we found that LIF was expressed in subsets of ISCs and Paneth cells, as well as some TA cells (FIGS. 1E & 1F). Taken together, these results demonstrate that LIF is expressed in intestinal crypt epithelial cells.

### Example 3 LIF Deficiency Impairs Development of the Small Intestine in Mice

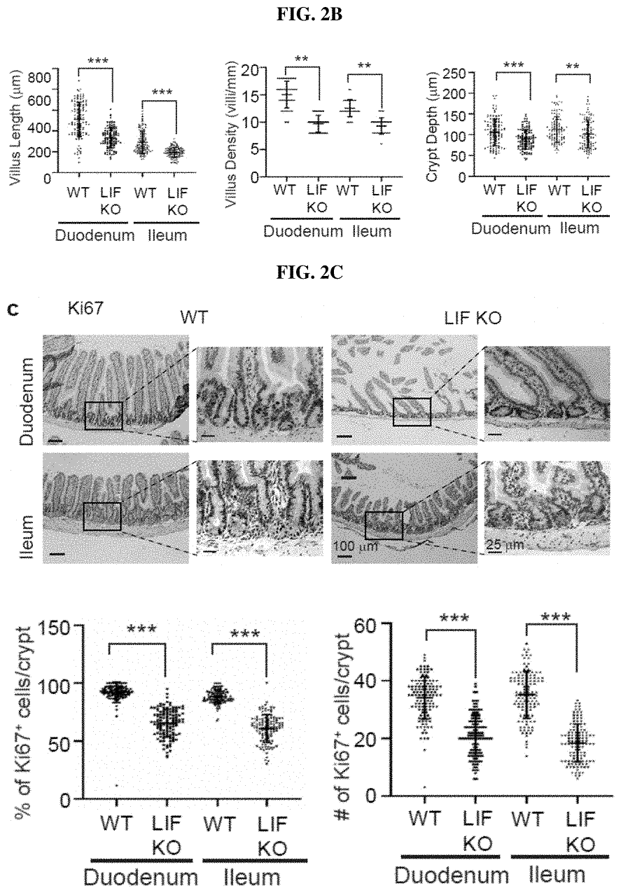

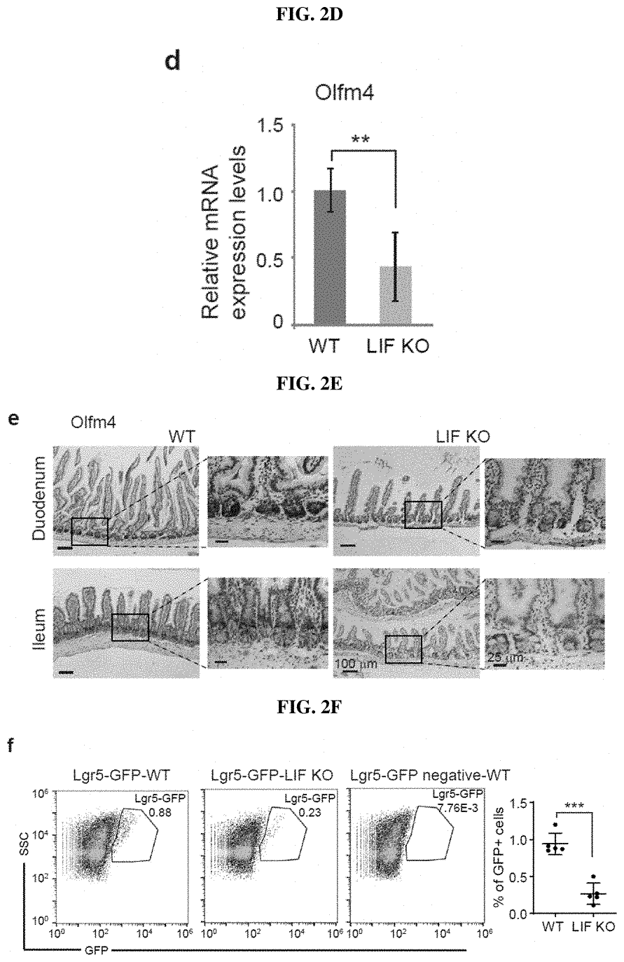

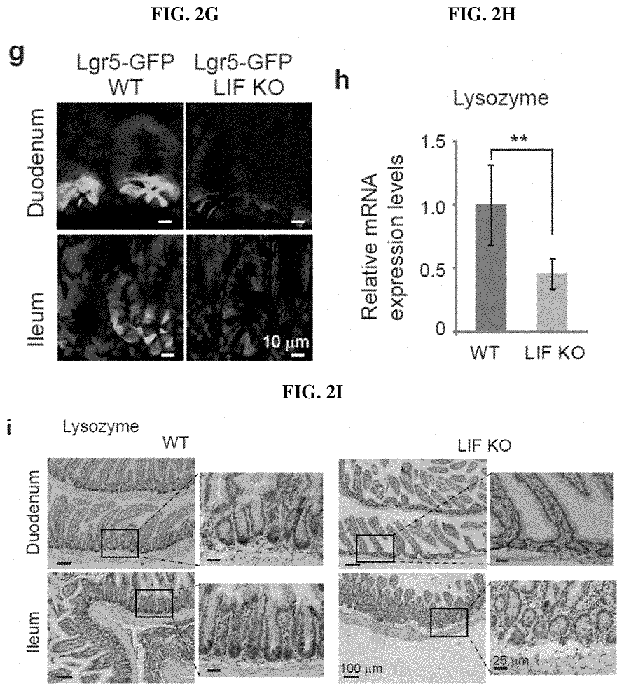

While it has been well-established that LIF is essential for the maintenance of pluripotency of mouse embryonic stem cells, the role of LIF in regulation of adult ISCs is unclear. Breeding of mice heterozygous for LIF to generate LIF KO mice showed that while E14.5-day embryos exhibited a Mendelian distribution of LIF KO allele, offspring mice at the weaning-age exhibited a non-Mendelian inheritance of the LIF KO allele; homozygous LIF KO offspring mice were fewer than the predicted ratios (Table 1). Viable LIF KO mice showed a retarded postnatal growth rate which resulted in ˜20% lower body weights compared with WT mice, which is consistent with a previous study (19). Histological analysis of the small intestine of LIF KO mice revealed that the length and the density of intestinal villi in LIF KO mice were significantly reduced compared with WT mice (FIGS. 2A & 2B), although different differentiated cell types, including goblet and tuft cells, were present in the intestinal villi in LIF KO mice. In addition to the villus blunting, a marked decrease in the crypt size along the entire small intestine and colon was detected in LIF KO mice (FIGS. 2A & 2B). The villus and crypt length was normalized to mouse body weight. Therefore, their significant differences between WT and LIF KO mice were unlikely due to the secondary effects caused by the growth delay of LIF KO mice. These results suggest that LIF is essential for intestinal epithelial homeostasis. We further examined intestinal epithelial proliferation by IHC staining of Ki67. In the small intestine of WT mice, Ki67 positive (Ki67⁺) cells were identified from position +4 counting from the bottom of the crypt and upwards to mark the TA population (20). LIF deficiency led to a significant decrease in this proliferative population in the small intestine in terms of both the number and the percentage of Ki67⁺ cells per crypt (FIG. 2C). However, LIF deficiency did not have an apparent effect on apoptosis of the intestinal epithelium as determined by IHC staining of cleaved caspase-3. These results indicate that the epithelial phenotypes observed in the intestine of LIF KO mice are likely due to an impaired epithelial proliferation rather than increased apoptosis.

To investigate the effect of LIF deficiency on ISCs, the expression of ISC marker Olfm4 was examined at both RNA and protein levels by quantitative real-time PCR and IHC staining assays, respectively. Compared with WT mice, Olfm4 mRNA and protein levels were dramatically reduced in the crypt of LIF KO mice (FIGS. 2D & 2E). The Lgr5-EGFP-IRES-creERT2 mice (namely Lgr5-GFP mice) with a knock-in allele expressing GFP from the Lgr5 locus have been widely used for the identification of ISC population (21). We used Lgr5-GFP mice to re-examine ISCs in the crypt in LIF KO mice. LIF KO; Lgr5-GFP mice displayed a reduced number of Lgr5-GFP positive cells and reduced GFP protein levels in remaining positive cells in crypts from the small intestine as determined by flow cytometry assays and IF staining, respectively (FIGS. 2F & 2G). Similarly, LIF KO; Lgr5-GFP mice displayed a reduced number of Lgr5-GFP positive cells in crypts from the colon. Furthermore, the levels of CD44, another ISC marker (22), were greatly reduced in the crypt of LIF KO mice compared with WT mice.

Paneth cells constitute the niche for stem cells in the intestinal crypt and are the major supportive epithelial cells for ISCs (23). Compared with WT mice, the levels of lysozyme, a Paneth cell marker, were clearly reduced in the intestinal tissue of LIF KO mice at both mRNA and protein levels (FIGS. 2H & 2I). Furthermore, LIF KO mice displayed an overall decreased fluorescence intensity of lysozyme staining and a significantly reduced Paneth cell count per crypt. Collectively, our results show that LIF deficiency leads to the decreased number of Lgr5⁺ISCs and Paneth cells in crypts and reduced proliferation of intestinal epithelium cells, which impairs the homeostasis of self-renewing small intestinal crypts.

### Example 4 LIF Deficiency Impairs Growth of the Intestinal Organoids

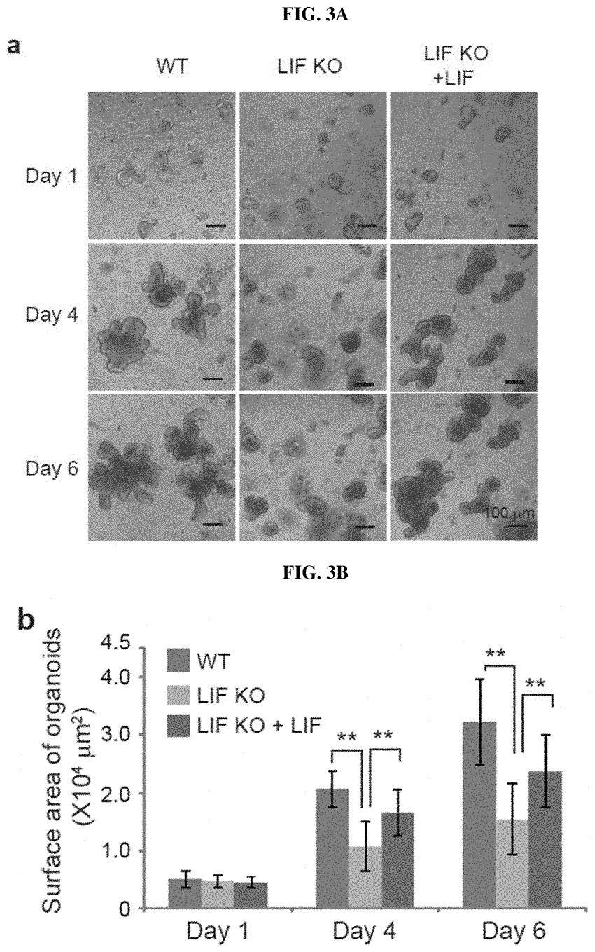

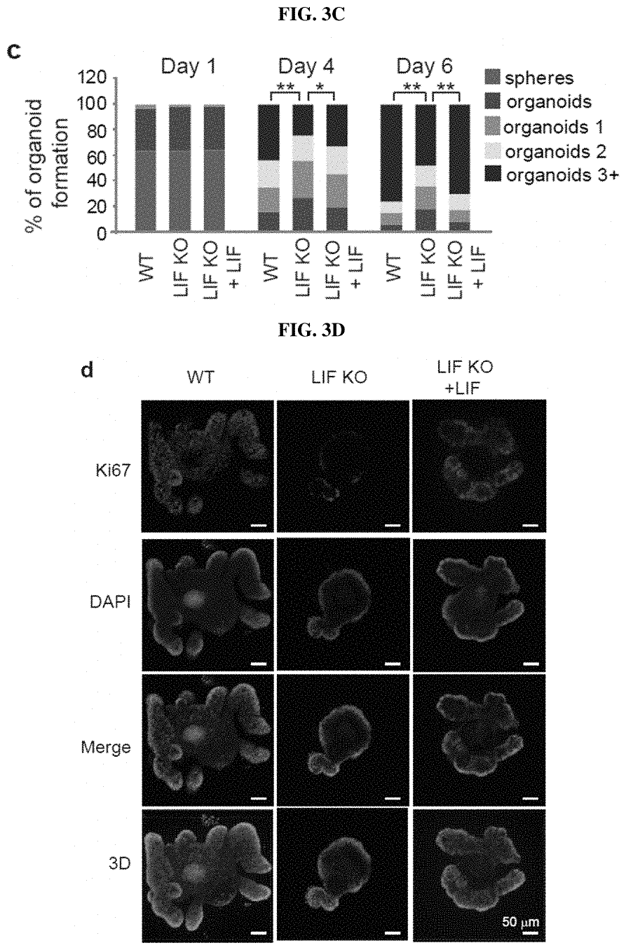

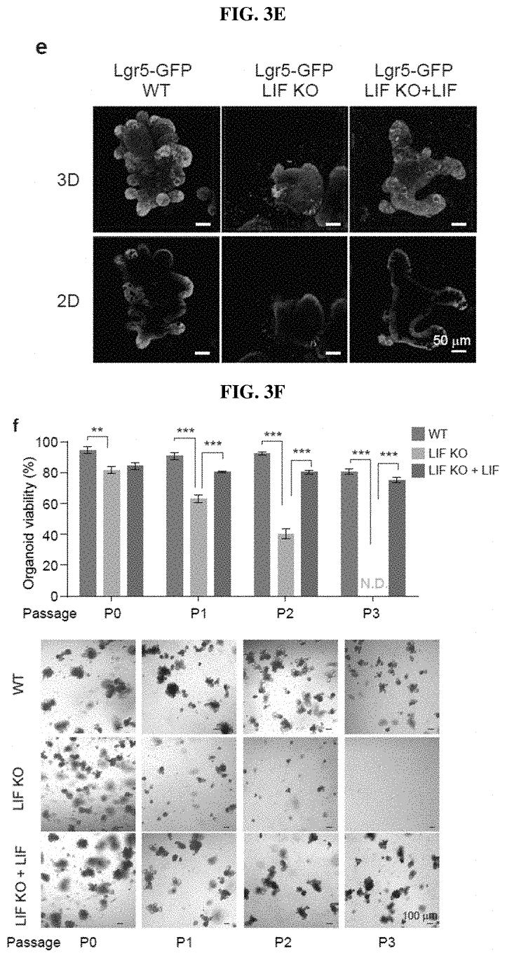

Next, we examined whether LIF regulates the function of ISC compartment by employing an organoid model of ex vivo epithelial regeneration (24). Compared with WT crypts, LIF KO crypts exhibited a significantly reduced ability to proliferate, expand and form budding organoids (FIG. 3A-3C). The growth of LIF KO organoids was much slower than WT organoids, with significantly reduced epithelial buds (FIG. 3A-2C). The average surface area of LIF KO organoids was ˜50% of that of WT organoids (FIG. 3B). Notably, the impaired growth of LIF KO organoids was largely rescued by supplementing culture media with recombinant mouse LIF (FIG. 3A-3C). Similarly, the number of Ki67⁺ cells was greatly decreased in LIF KO organoids, which was largely restored by the supplement of recombinant mouse LIF (FIG. 3D). The number of Paneth cells as determined by IHC staining of UEA-1, a Paneth cell marker (25), was also decreased in LIF KO organoids, which was largely restored by the supplement of recombinant mouse LIF Organoids formed from LIF KO; Lgr5-GFP mouse crypts displayed dramatically decreased Lgr5-GFP levels compared with WT organoids as determined by IF staining, and the reduction was largely rescued by the supplement of recombinant mouse LIF (FIG. 3E). Unlike WT organoids, which grow indefinitely, LIF KO organoids had a lower survival rate after passaging, and died out after 3 passages, indicating that LIF KO organoids had a reduced self-renewal capacity compared with WT organoids (FIG. 3F). Notably, the impaired self-renewal ability of LIF KO organoids was largely rescued by supplementing culture media with recombinant mouse LIF (FIG. 3F). Taken together, these results demonstrate an important role of LIF in maintaining the clonogenic activity.

### Example 5 LIF Effects on β-Catenin Signaling in the Small Intestine

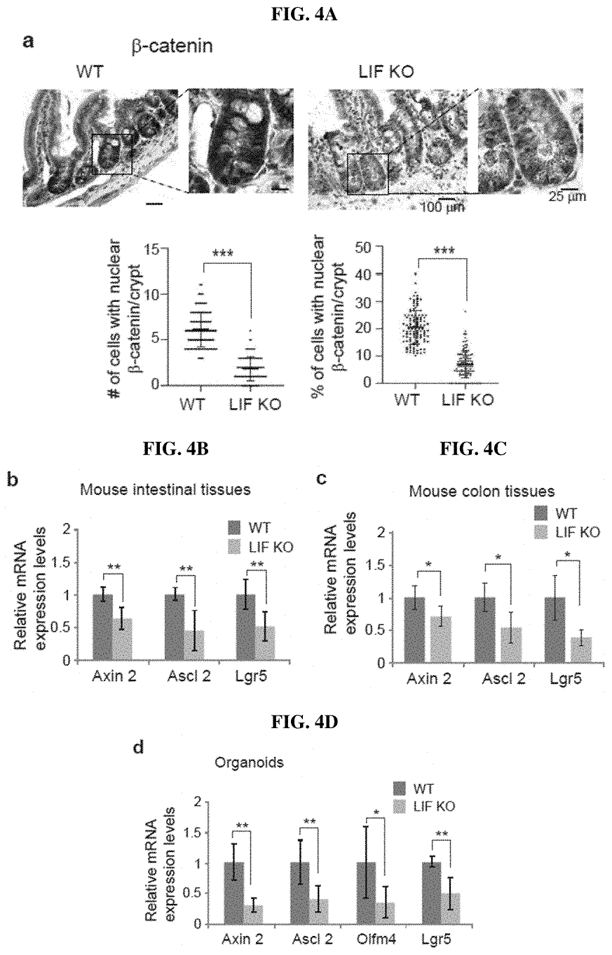

Proper β-catenin signaling is critical for ISC function and intestinal crypt homeostasis (15, 16). The nuclear accumulation of β-catenin at the bottom of crypts in the small intestine functions as a co-activator of TCF/LEF proteins to regulate the expression of a group of genes, and thus plays a crucial role in maintaining crypt stem/progenitor cell compartments and the homeostasis of intestinal epithelium (15, 16). Therefore, we investigated whether LIF regulates the β-catenin signaling to maintain ISC function and intestinal crypt homeostasis. IHC assays showed that LIF deficiency significantly reduced nuclear β-catenin accumulation in crypts in the small intestines (FIG. 4A). Quantitative real-time PCR assays showed that mRNA levels of a panel of well-known β-catenin/TCF target genes, including Axin2, Ascl1 and Lgr5 (26), were significantly decreased in the small intestine and colon of LIF KO mice compared with those of WT mice (FIGS. 4B & 4C). Olfm4 is also a β-catenin target gene, which is expressed in the small intestine but not in the colon of mice (27). The mRNA levels of Olfm4 were also significantly decreased in the small intestine of LIF KO mice (FIG. 2D). Similar reductions in mRNA levels of Axin2, Ascl1, Olfm4 and Lgr5 were observed in LIF KO organoids compared with WT organoids (FIG. 4D). Together, these results indicate that LIF deficiency decreases the nuclear accumulation and transcriptional activity of β-catenin in crypts, which in turn impairs ISC function and homeostasis of the intestinal epithelium.

### Example 6 LIF Upregulates the β-Catenin Signaling Via the AKT/GSK3β Signaling in the Small Intestine

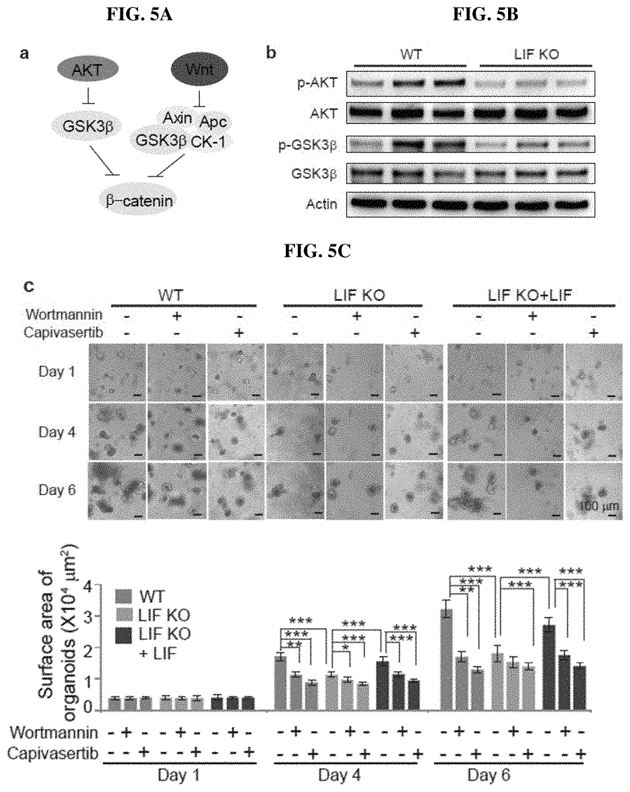

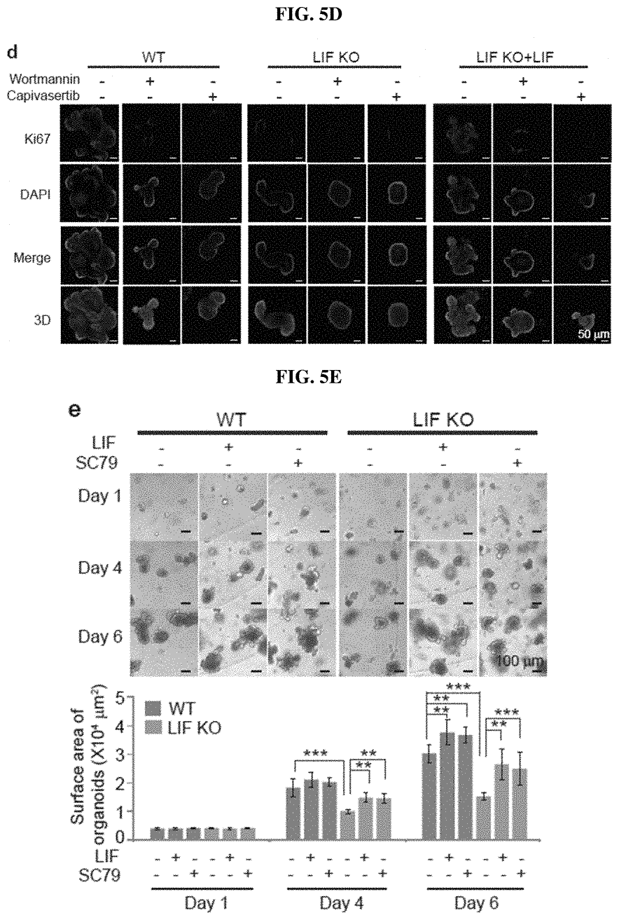

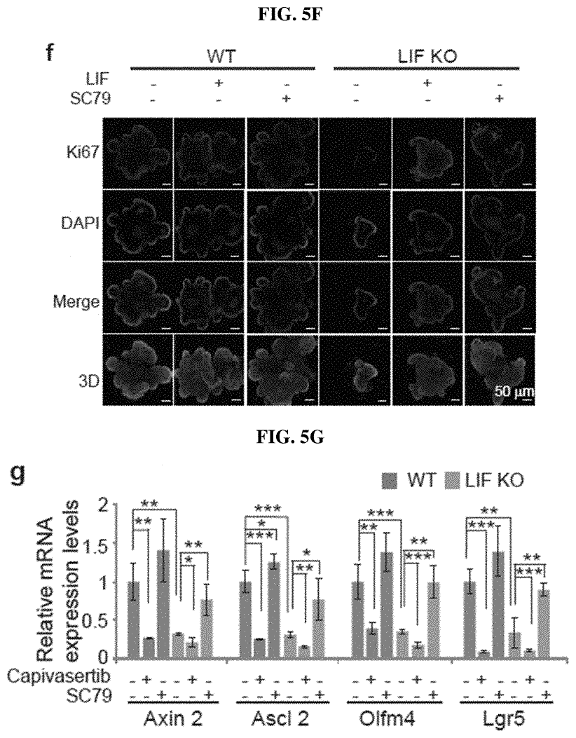

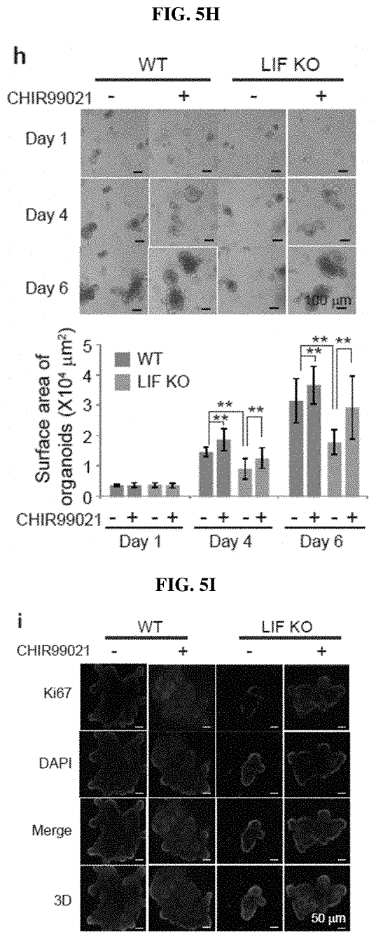

AKT is an important downstream target of LIF, which plays a critical role in mediating many important functions of LIF (28, 29). Currently, it is unclear whether the LIF/AKT signaling is involved in the regulation of ISC function and homeostasis of the intestinal epithelium. AKT has been reported to phosphorylate GSK3β at Ser-9 to inactivate GSK3β in different types of cells, which in turn stabilizes β-catenin (30) (FIG. 5A).

Interestingly, we found that LIF deficiency greatly decreased the AKT activity in the small intestine as reflected by the decreased levels of AKT phosphorylation at Ser-473 (p-AKT) in the small intestine of LIF KO mice compared with that of WT mice (FIG. 5B). LIF deficiency also greatly decreased the levels of GSK3β phosphorylation at Ser-9 in the small intestine (FIG. 5B), indicating that LIF deficiency leads to increased GSK3β activity to promote β-catenin degradation and inhibit its function.

To investigate whether LIF regulates ISC function through upregulating the AKT signaling, WT and LIF KO organoids were treated with Wortmannin, a PI3K inhibitor that inhibits the PI3K/Akt signaling, and Capivasertib, a specific AKT inhibitor (31, 32). Both Wortmannin and Capivasertib treatments greatly inhibited the growth of WT organoids and decreased cell proliferation as determined by analyzing the surface area of organoids, percentage of organoid formation, and number of Ki67⁺ cells, respectively, at the end of treatment (FIGS. 5C & 5D). In contrast, the inhibitory effects of Wortmannin and Capivasertib on organoid growth and cell proliferation were much less pronounced in LIF KO organoids which displayed a reduced AKT activity and impaired growth (FIGS. 5C & 5D). Notably, while supplementation of recombinant mouse LIF largely rescued the impaired growth of LIF KO organoids, Wortmannin and Capivasertib treatments largely abolished the rescue effect of recombinant LIF on the growth of LIF KO organoids (FIGS. 5C & 5D). SC79, an AKT agonist, greatly improved the growth and cell proliferation of LIF KO organoids but displayed a less pronounced effect on WT organoids (FIGS. 5E & 5F). Notably, SC79 appeared to rescue the impaired growth of LIF KO organoids to a similar extent as recombinant mouse LIF did (FIGS. 5E & 5F). Further, we directly examined the effects of Capivasertib and SC79 on β-catenin activity by measuring the mRNA levels of its target genes in WT and LIF KO organoids. Capivasertib treatment significantly decreased the mRNA levels of Axin2, Ascl1, Olfm4 and Lgr5 in WT organoids but displayed a less pronounced effect on these genes in LIF KO organoids. SC79 greatly increased the mRNA levels of these genes in LIF KO organoids but displayed a less pronounced effect on them in WT organoids (FIG. 5G). We further treated organoids formed by WT or LIF KO; Lgr5-GFP mouse crypts with Wortmannin, Capivasertib or SC79. Wortmanin and Capivasertib greatly decreased stem cell number in WT organoids and exhibited a much less pronounced inhibitory effect on LIF KO organoids, and SC79 greatly increased stem cell number in LIF KO organoids but showed a less pronounced effect on WT organoids (FIG. 10). Together, these results suggest that LIF regulates β-catenin activity and promotes organoid growth mainly through AKT activation. We further examined whether blocking GSK3β can improve the growth of LIF KO organoids. CHIR99021, a specific GSK3β inhibitor (33), greatly improved the growth, cell proliferation and stem cell number of LIF KO organoids but showed a much less pronounced effect on WT organoids (FIGS. 5H & 5I).

In addition to the AKT signaling, LIF can activate multiple signaling pathways, including JAK/STAT3 and MAPK pathways, to mediate some of LIF's functions. Here, we investigated whether these two signaling pathways are involved in the regulation of LIF-dependent intestinal * homeostasis by treating WT and LIF KO organoids with Stattic and SB242235, inhibitors for STAT3 and MAPK, respectively (34, 35). Blocking STAT3 or MAPK signaling pathway did not have a significant effect on the growth and proliferation of WT or LIF KO organoids (FIG. 10). Furthermore, combined treatment of Wortmannin and Stattic did not show a stronger inhibitory effect on the growth and proliferation of WT or LIF KO organoids than Wortmannin treatment alone. These results suggest that JAK/STAT3 and MAPK pathways do not play a major role in LIF's function of maintaining ISC function.

It has been reported that β-catenin can be upregulated by Wnt ligands in the small intestine (15). Wnt ligands bind to receptors composed of Frizzled receptors and LRP5/6 to inactivate a multi-protein complex for β-catenin degradation, leading to the release of β-catenin from this multi-protein complex (36). Thus, β-catenin becomes stabilized and translocates into the nucleus (FIG. 5A). Wnt 3 is a major Wnt ligand in small intestinal epithelial cells (37, 38). In addition, EGF and D111 are key ligands that provide support to Lgr5⁺ ISCs. However, we found that LIF deficiency did not significantly affect the mRNA levels of Wnt 3, EGF and D111 in the small intestine as determined by quantitative real-time PCR assays.

Collectively, the results herein strongly suggest that LIF upregulates the β-catenin signaling mainly through the AKT/GSK3β signaling in the small intestine to maintain ISC function.

### Example 7. LIF Deficiency Impairs Intestinal Epithelial Regeneration and Reduces Lifespan after Radiation in Mice

Normal function of ISCs is critical for intestinal regeneration after injury, such as IR-induced epithelial renewal (39). It has been reported that in response to high doses of IR (such as 12 Gy whole-body IR), the intestinal epithelium of mice goes through an apoptotic phase with an increase in crypt cell apoptosis, a massive loss of crypt cells and a shortening of crypts and villi in the first 2 days, which is followed by a proliferative phase showing regeneration of crypts by surviving crypt cells (10). Due to the interruption of the extremely rapid cell turnover and lose of sufficient crypts in the intestine, proper intestinal mucosal barrier can no longer be maintained, which is susceptible to infection. Eventually the intestine becomes ulcerated, a phenomena called GI syndrome, which leads to death of mice within days (40).

Here, we investigated the role of LIF in IR-induced intestinal epithelial regeneration in mice. We found that the impairment of ISC function in LIF KO mice was exacerbated by 12 Gy whole-body IR. While the small intestines of WT mice showed mild villous blunting at 72 h after IR, the small intestine of LIF KO mice showed more severe epithelial injury, including moderate to severe villous blunting, less lamina propria, disorganized villous architecture in ileum, and immature intestinal epithelium which was marked by loss of goblet cells (FIG. 6A). In WT mice, numerous enlarged/hyperplastic crypts indicative of regeneration were observed at 72 h after IR (FIG. 6A). In contrast, regeneration was impaired with a dramatic reduction of regenerating crypts in LIF KO mice at 72 h after IR (FIG. 6A). At 72 h after IR, compared with the small intestine of WT mice, the small intestine of LIF KO mice contained significantly fewer viable crypts (defined as a crypt-like structure containing at least five adjacent Ki67⁺ cells) (FIGS. 6B & 6C), and significantly lower levels of Olfm4 (FIGS. 6D & 6E). Notably, the impairment of intestinal regeneration in LIF KO mice was largely rescued by administering mice with recombinant mouse LIF (i.p. 30 ng/g body weight, twice a day for 7 days, starting from 3 days before IR); there was normal appearing small intestinal epithelium, suggesting remarkable epithelial recovery in the small intestine in these mice (FIG. 6A). Importantly, administering mice with recombinant mouse LIF greatly increased the number of viable crypts and Olfm4 levels in the small intestine of LIF KO mice to a similar extent as observed in WT mice (FIG. 6B-6E).

Consistent with previous reports (41, 42), WT mice subjected to 12 Gy whole-body IR had a median lifespan of 9 days due to the GI syndrome (FIG. 6F). Compared with WT mice, LIF KO mice subjected to 12 Gy IR had a significantly reduced lifespan with a median lifespan of 7 days (p<0.001) (FIG. 6F). Notably, this reduction of lifespan can be restored by administering recombinant LIF to LIF KO mice (FIG. 6F). These results demonstrate that LIF deficiency impairs intestine epithelial regeneration and reduces lifespan in mice after IR, which can be rescued by administering recombinant LIF, indicating that LIF is important for efficient regeneration of the intestinal epithelium, as well as survival of mice upon injury challenge.

### Example 8 Administering Recombinant LIF Promotes the Regeneration of Intestinal Epithelium and Prolongs Lifespan after Radiation in WT Mice

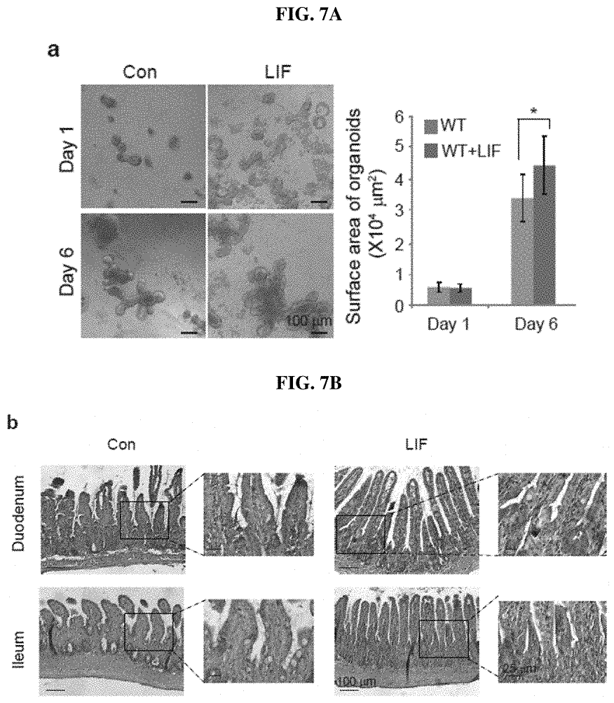

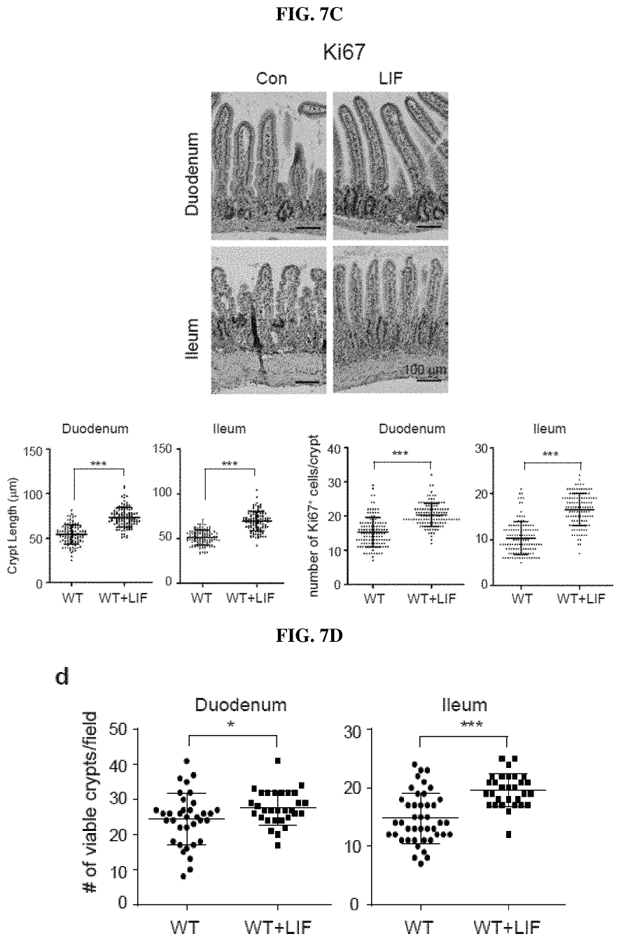

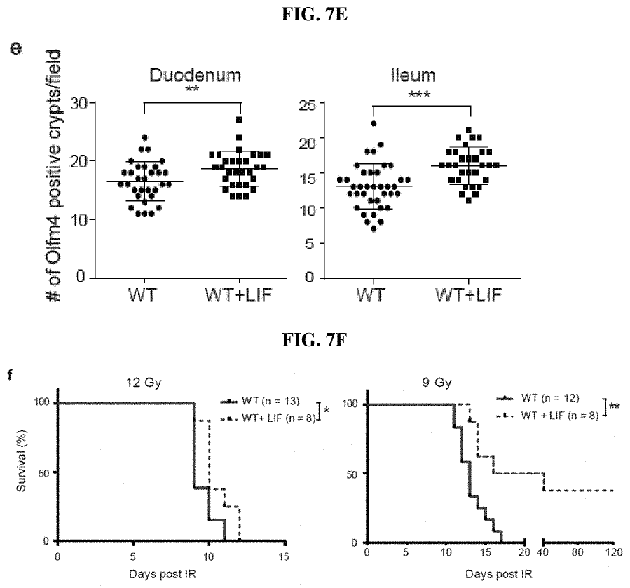

We next investigated whether supplementing LIF can improve adult ISC function and protect against radiation-induced GI syndrome in WT mice. We first examined the effect of supplementing LIF on the growth of WT intestinal organoids. LIF supplementation in culture media promoted the growth of organoids with enlarged surface area (FIG. 7A), suggesting that LIF enhances the clonogenic activity of WT ISCs. We further examined whether supplementing LIF has a radioprotective effect on WT mice through promoting the regeneration of intestinal epithelium. Recombinant mouse LIF was administered to WT mice via i.p. at a dose of 30 ng/g body weight, twice a day for 7 days, starting from 3 days before IR. At 72 h after 12 Gy IR, WT mice with LIF treatment showed much greater intestinal regeneration compared with the control mice treated with vehicle. LIF treatment increased the length and density of villi and the number of viable crypts in the intestinal tissues of WT mice (FIG. 7B). Compared with control mice treated with vehicle, the average length of crypts was much longer, and the average number of proliferating cells which are Ki67 positive in each crypt was significantly higher in WT mice administrated with LIF (FIG. 7C), indicating that LIF enhanced ISC regeneration function. The numbers of viable crypts and Olfm4 levels were also increased in WT mice with LIF treatment, compared with the control mice (FIGS. 7D & 7E). Furthermore, LIF administration significantly prolonged the lifespan of WT mice in response to 12 Gy whole-body IR; LIF-treated mice had a median survival of 10 days whereas control mice had a median survival of 9 days (p=0.03) (FIG. 7F). The protective effect was more obvious when WT mice were subject to 9 Gy whole-body irradiation; while the control group had a median lifespan of 13 days, LIF-treated group had a median lifespan of 28.5 days with ˜50% of the group bypassed GI syndrome-induced lethality (p=0.005) (FIG. 7F). These results demonstrate a radioprotective role of LIF; administering recombinant LIF promotes intestinal epithelial regeneration and prolongs survival of mice after irradiation.

### Example 9. Effect of LIF Overexpression on the Regeneration of Intestinal Epithelium after Irradiation in Inducible LIF Transgenic (LIF-tg^{flox/+}/Cre-ERT2) Mice

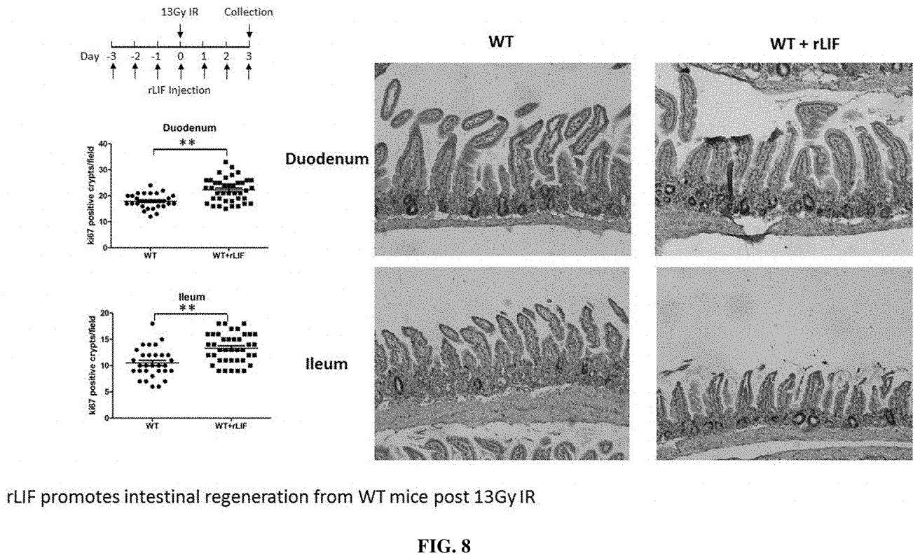

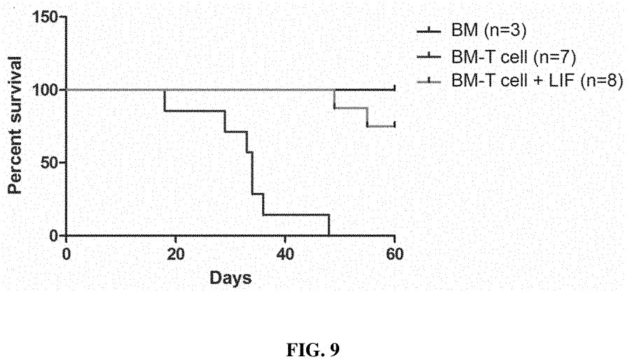

Eight-twelve-week-old male and female LIF-tg^{flox/+}/Cre-ERT2 mice were injected (i.p.) with tamoxifen (80 ng/g body weight for male and 40 ng/g body weight for female) to induce LIF expression 3 days before irradiation (D-3). Age- and gender-matched LIF-tg^{flox/+}/Cre-ERT2 mice were injected with corn oil as control on D-3. On D0 mice were subject to 12 Gy-whole-body irradiated. Mouse intestine tissue was collected at 72 h after irradiation (D3). The levels of Ki67 in intestinal epithelium were determined by immunohistochemistry (IHC) staining. n=6 in Tamoxifen treated group, n=4 in control group. The results in FIG. 8 show that tamoxifen induced increase in LIF expression resulted in significantly higher intestinal crypt regeneration (˜50% higher), compared to controls, after irradiation.

### Example 10 LIF Pretreatment Reduces the Severity of GvHD Following Bone Marrow Transplant

Graft versus host disease (GvHD) was established using a MHC haplo-mismatch transplantation model. After pre-conditioning with ionized radiation, recipient mice received bone marrow (BM) transplantation with or without allogeneic donor T cells. While recipient mice receiving BM transplantation without T cells appeared normal throughout lifespan, those receiving allogeneic donor T cells (BM-T cell) started to show body weight loss, followed by diarrhea, hunching posture, dull fur, and imparted movement, which are characteristics of murine models of GvHD, within weeks after BMT, suggesting success GvHD induction. These mice that developed GvHD showed a medium life span of ˜30 days. To test whether LIF protects mice from GvHD, recombinant mouse LIF protein was injected into recipient mice. Mouse body weight, health condition, and survival were monitored. Results indicate that LIF treatment greatly reduced the development of GvHD and prolonged the survival of mice.

Alternatives and Extensions

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items. elements or steps. Furthermore, the indefinite article “a” or “an” is meant to indicate one or more of the item, element or step modified by the article.

### REFERENCES

The entire contents of the following references are hereby incorporated by reference as if fully set forth herein, except for terminology that is inconsistent with the terminology used herein.
1. Mathieu M E, Saucourt C, Mournetas V, Gauthereau X, Theze N, Praloran V, et al. LIFdependent signaling: new pieces in the Lego. Stem Cell Rev. 2012; 8(1):1-15.
2. Yue X, Wu L, Hu W. The regulation of leukemia inhibitory factor. Cancer Cell Microenviron. 2015; 2(3).
3. Hu W, Feng Z, Teresky A K, Levine A J. p53 regulates maternal reproduction through LIF. Nature. 2007; 450(7170):721-4.
4. Kang H J, Feng Z, Sun Y, Atwal G, Murphy M E, Rebbeck T R, et al. Single-nucleotide polymorphisms in the p53 pathway regulate fertility in humans. Proc Natl Acad Sci USA. 2009; 106(24):9761-6.
5. Onishi K, Zandstra P W. LIF signaling in stem cells and development. Development. 2015; 142(13):2230-6.
6. Niwa H, Ogawa K, Shimosato D, Adachi K. A parallel circuit of LIF signalling pathways maintains pluripotency of mouse E S cells. Nature. 2009; 460(7251):118-22.
7. Tang M-s, Wang H-t, Hu Y, Chen W-S, Akao M, Feng Z, et al. Acrolein induced DNA damage, mutagenicity and effect on DNA repair. Molecular Nutrition & Food Research. 2011; 55(9):1291-300.
8. Moore K A, Lemischka I R. Stem cells and their niches. Science. 2006; 311(5769):1880-5.
9. Clevers H. The intestinal crypt, a prototype stem cell compartment. Cell. 2013; 154(2):274-84.
10. Kim C K, Yang V W, Bialkowska A B. The Role of Intestinal Stem Cells in Epithelial Regeneration Following Radiation-Induced Gut Injury. Curr Stem Cell Rep. 2017; 3(4):320-32.
11. Tian H, Biehs B, Warming S, Leong K G, Rangell L, Klein O D, et al. A reserve stem cell population in small intestine renders Lgr5-positive cells dispensable. Nature. 2011; 478(7368):255-9.
12. Tetteh P W, Basak O, Farin H F, Wiebrands K, Kretzschmar K, Begthel H, et al. Replacement of Lost Lgr5-Positive Stem Cells through Plasticity of Their Enterocyte-Lineage Daughters. Cell Stem Cell. 2016; 18(2):203-13.
13. Westphalen C B, Asfaha S, Hayakawa Y, Takemoto Y, Lukin D J, Nuber A H, et al. Long-lived intestinal tuft cells serve as colon cancer-initiating cells. J Clin Invest. 2014; 124(3):1283-95.
14. Yu S, Tong K, Zhao Y, Balasubramanian I, Yap G S, Ferraris R P, et al. Paneth Cell Multipotency Induced by Notch Activation following Injury. Cell Stem Cell. 2018; 23(1):46-59 e5.
15. Kretzschmar K, Clevers H. Wnt/beta-catenin signaling in adult mammalian epithelial stem cells. Dev Biol. 2017; 428(2):273-82.
16. MacDonald B T, Tamai K, He X. Wnt/beta-catenin signaling: components, mechanisms, and diseases. Dev Cell. 2009; 17(1):9-26.
17. Fevr T, Robine S, Louvard D, Huelsken J. Wnt/beta-catenin is essential for intestinal homeostasis and maintenance of intestinal stem cells. Mol Cell Biol. 2007; 27(21):7551-9.
18. Feng Q, Gao N. Keeping Wnt signalosome in check by vesicular traffic. J Cell Physiol. 2015; 230(6):1170-80.
19. Stewart C L, Kaspar P, Brunet U, Bhatt H, Gadi I, Kontgen F, et al. Blastocyst implantation depends on maternal expression of leukaemia inhibitory factor. Nature. 1992; 359(6390):76-9.
20. Spit M, Koo B K, Maurice M M. Tales from the crypt: intestinal niche signals in tissue renewal, plasticity and cancer. Open Biol. 2018; 8(9).
21. Barker N, van Es J H, Kuipers J, Kujala P, van den Born M, Cozijnsen M, et al. Identification of stem cells in small intestine and colon by marker gene Lgr5. Nature. 2007; 449(7165):1003-7.
22. Gracz A D, Fuller M K, Wang F, Li L, Stelzner M, Dunn J C, et al. Brief report: CD24 and CD44 mark human intestinal epithelial cell populations with characteristics of active and facultative stem cells. Stem Cells. 2013; 31(9):2024-30.
23. Sato T, van Es J H, Snippert H J, Stange D E, Vries R G, van den Born M, et al. Paneth cells constitute the niche for Lgr5 stem cells in intestinal crypts. Nature. 011; 469(7330):415-8.
24. Sato T, Vries R G, Snippert H J, van de Wetering M, Barker N, Stange D E, et al. Single Lgr5 stem cells build crypt-villus structures in vitro without a mesenchymal niche. Nature. 2009; 459(7244):262-5.
25. Buczacki S J, Zecchini H I, Nicholson A M, Russell R, Vermeulen L, Kemp R, et al. Intestinal label-retaining cells are secretory precursors expressing Lgr5. Nature. 2013; 495(7439):65-9.
26. Herbst A, Jurinovic V, Krebs S, Thieme S E, Blum H, Goke B, et al. Comprehensive analysis of beta-catenin target genes in colorectal carcinoma cell lines with deregulated Wnt/beta-catenin signaling. BMC Genomics. 2014; 15:74.
27. Liu W, Li H, Hong S H, Piszczek G P, Chen W, Rodgers G P. Olfactomedin 4 deletion induces colon adenocarcinoma in Apc(Min/+) mice. Oncogene. 2016; 35(40):5237-47.
28. Li X, Yang Q, Yu H, Wu L, Zhao Y, Zhang C, et al. LIF promotes tumorigenesis and metastasis of breast cancer through the AKT-mTOR pathway. Oncotarget. 2014; 5(3):788-801.
29. Ohtsuka S, Nakai-Futatsugi Y, Niwa H. LIF signal in mouse embryonic stem cells. JAKSTAT 2015; 4(2):e1086520.
30. Cross D A, Alessi D R, Cohen P, Andjelkovich M, Hemmings B A. Inhibition of glycogen synthase kinase-3 by insulin mediated by protein kinase B. Nature. 1995; 378(6559):785-9.
31. Wymann M P, Bulgarelli-Leva G, Zvelebil M J, Pirola L, Vanhaesebroeck B, Waterfield M D, et al. Wortmannin inactivates phosphoinositide 3-kinase by covalent modification of Lys-802, a residue involved in the phosphate transfer reaction. Mol Cell Biol. 1996; 16(4):1722-33.
32. Davies B R, Greenwood H, Dudley P, Crafter C, Yu D H, Zhang J, et al. Preclinical pharmacology of AZD5363, an inhibitor of AKT: pharmacodynamics, antitumor activity, and correlation of monotherapy activity with genetic background. Mol Cancer Ther. 2012; 11(4):873-87.
33. Ring D B, Johnson K W, Henriksen E J, Nuss J M, Goff D, Kinnick T R, et al. Selective glycogen synthase kinase 3 inhibitors potentiate insulin activation of glucose transport and utilization in vitro and in vivo. Diabetes. 2003; 52(3):588-95.
34. Foster M L, Halley F, Souness J E. Potential of p38 inhibitors in the treatment of rheumatoid arthritis. Drug News Perspect. 2000; 13(8):488-97.
35. Schust J, Sperl B, Hollis A, Mayer T U, Berg T. Stattic: a small-molecule inhibitor of STAT3 activation and dimerization. Chem Biol. 2006; 13(11):1235-42.
36. MacDonald B T, He X. Frizzled and LRP5/6 receptors for Wnt/beta-catenin signaling. Cold Spring Harb Perspect Biol. 2012; 4(12).
37. Sasaki N, Sachs N, Wiebrands K, Ellenbroek S I, Fumagalli A, Lyubimova A, et al. Reg4 deep crypt secretory cells function as epithelial niche for Lgr5+ stem cells in colon. Proc Natl Acad Sci USA. 2016; 113(37):E5399-407.
38. Perochon J, Carroll L R, Cordero J B. Wnt Signalling in Intestinal Stem Cells: Lessons from Mice and Flies. Genes (Basel). 2018; 9(3).
39. Metcalfe C, Kljavin N M, Ybarra R, de Sauvage F J. Lgr5+ stem cells are indispensable for radiation-induced intestinal regeneration. Cell Stem Cell. 2014; 14(2):149-59.
40. Macia IGM, Lucas Calduch A, Lopez E C. Radiobiology of the acute radiation syndrome. Rep Pract Oncol Radiother. 2011; 16(4):123-30.
41. Booth C, Tudor G, Tudor J, Katz B P, MacVittie T J. Acute gastrointestinal syndrome in highdose irradiated mice. Health Phys. 2012; 103(4):383-99.
42. Kirsch D G, Santiago P M, di Tomaso E, Sullivan J M, Hou W S, Dayton T, et al. p53 controls radiation-induced gastrointestinal syndrome in mice independent of apoptosis. Science. 2010; 327(5965):593-6.
43. Moreau J F, Donaldson D D, Bennett F, Witek-Giannotti J, Clark S C, Wong G G. Leukaemia inhibitory factor is identical to the myeloid growth factor human interleukin for D A cells. Nature. 1988; 336(6200):690-2.
44. Hirai H, Karian P, Kikyo N. Regulation of embryonic stem cell self-renewal and pluripotency by leukaemia inhibitory factor. Biochem J. 2011; 438(1):11-23.
45. Martello G, Smith A. The nature of embryonic stem cells. Annu Rev Cell Dev Biol. 2014; 30:647-75.
46. Boeuf H, Hauss C, Graeve F D, Baran N, Kedinger C. Leukemia inhibitory factor-dependent transcriptional activation in embryonic stem cells. J Cell Biol. 1997; 138(6):1207-17.
47. Niwa H, Burdon T, Chambers I, Smith A. Self-renewal of pluripotent embryonic stem cells is mediated via activation of STAT3. Genes Dev. 1998; 12(13):2048-60.
48. Zhang Y S, Xin D E, Wang Z, Song X, Sun Y, Zou Q C, et al. STAT4 activation by leukemia inhibitory factor confers a therapeutic effect on intestinal inflammation. EMBO J. 2019; 38(6).
49. Das S, Yu S, Sakamori R, Vedula P, Feng Q, Flores J, et al. Rab8a vesicles regulate Wnt ligand delivery and Paneth cell maturation at the intestinal stem cell niche. Development. 2015; 142(12):2147-62.
50. Zwarycz B, Gracz A D, Rivera K R, Williamson I A, Samsa L A, Starmer J, et al. IL22 Inhibits Epithelial Stem Cell Expansion in an Ileal Organoid Model. Cell Mol Gastroenterol Hepatol. 2019; 7(1):1-17.
51. Farin H F, Van Es J H, Clevers H. Redundant sources of Wnt regulate intestinal stem cells and promote formation of Paneth cells. Gastroenterology. 2012; 143(6):1518-29 e7.
52. Gracz A D, Puthoff B J, Magness S T. Identification, isolation, and culture of intestinal epithelial stem cells from murine intestine. Methods Mol Biol. 2012; 879:89-107.
53. Zhao Y, Wu L, Yue X, Zhang C, Wang J, Li J, et al. A polymorphism in the tumor suppressor p53 affects aging and longevity in mouse models. Elife. 2018; 7.
54. Yue X, Zhang C, Zhao Y, Liu J, Lin A W, Tan V M, et al. Gain-of-function mutant p53 activates small GTPase Rac1 through SUMOylation to promote tumor progression. Genes Dev. 2017; 31(16):1641-54.
55. Wrzosek L, Miguel S, Noordine M L, Bouet S, Joncquel Chevalier-Curt M, Robert V, et al. Bacteroides thetaiotaomicron and Faecalibacterium prausnitzii influence the production of mucus glycans and the development of goblet cells in the colonic epithelium of a gnotobiotic model rodent. BMC Biol. 2013; 11:61.
56. Gao N, Kaestner K H. Cdx2 regulates endo-lysosomal function and epithelial cell polarity. Genes Dev. 2010; 24(12):1295-305.
57. Yu H, Yue X, Zhao Y, Li X, Wu L, Zhang C, et al. LIF negatively regulates tumour-suppressor p53 through Stat3/ID1/MDM2 in colorectal cancers. Nat Commun. 2014; 5:5218.
58. Hudson K R et al., J Biol Chem. 1996 May 17; 271(20):11971-8, Characterization of the receptor binding sites of human leukemia inhibitory factor and creation of antagonists.
59. Huyton T et al, Proc Natl Acad Sci USA. 2007 Jul. 31; 104(31):12737-42.
60. Gunawardana, et al., (2003) A phase I study of recombinant human leukemia inhibitory factor in patients with advanced cancer. Clin Cancer Res. June; 9(6):2056-65.
61. Wang H., Wang J., Zhao Y., Zhang X., Liu J., Zhang C., Haffty B., Verzi M., Zhang L., Gao N., Feng Z., Hu W. LIF is essential for ISC function and protects against radiation-induced gastrointestinal syndrome. In submission.
62. Yue X, Zhang C, Zhao Y, Liu J, Lin A W, Tan V M, Drake J M, Liu L, Boateng M N, Li J, Feng Z, Hu W. (2017) Gain-of-function mutant p53 activates small GTPase Rac1 through SUMOylation to promote tumor progression. Genes Dev. 31(16):1641-1654.
63. Lindemans, C. A., et al. (2015). Interleukin-22 promotes intestinal-stem-cell-mediated epithelial regeneration. Nature, 528(7583), 560.
64. Zhou, W. J., et al. (2013). Induction of intestinal stem cells by R-spondin 1 and Slit2 augments chemoradioprotection. Nature, 501(7465), 107.
65. Metcalf, D., et al. (1990); Effects of injected leukemia inhibitory factor on hematopoietic and other tissues in mice. Blood, 76(1), 50-56.)

What is claimed is:

1. A method for treating a subject suffering from gastrointestinal radiation injury (GRI) or graft vs. host disease (GvHD), or a subject at risk of developing GRI or GvHD, comprising administering a therapeutically effective amount of LIF or variant thereof or a biologically active fragment of LIF or variant thereof to treat the subject.

2. The method of claim 1, wherein the LIF or variant thereof or a biologically active fragment of LIF or variant thereof is administered as an enteric formulation, or is formulated for topical administration to an area of the intestine.

3. The method of claim 2, wherein the enteric formulation of LIF or variant thereof, or a biologically active fragment of LIF or a variant thereof is formulated for absorption by the small intestine.

4. The method of claim 3, wherein the enteric formulation of LIF or a variant thereof, or a biologically active fragment of LIF or a variant thereof is absorbed by the large intestine.

5. The method of claim 1, wherein the LIF or a variant thereof, or a biologically active fragment of LIF or a variant thereof is administered orally, subcutaneously, intravenously, intraperitoneally, rectally, topically to the intestine, parenterally, intraarterially, intrathecally, intradermally, buccally, sublingualy, or transmucosally.

6. The method of claim 1, wherein the effective amount of LIF or a variant thereof, or a biologically active fragment of LIF or a variant thereof administered per day is between 0.5 μg/kg and 100 μg/kg, between about 1 μg/kg and 75 μg/kg, between 0.25 μg/kg and 50 μg/kg, 5 μg/kg and 50 μg/kg, between 20 μg/kg and 40 μg/kg, or the amount is selected from the group consisting of 0.5 μg/kg, 1 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, and 60 μg/kg.

7. The method of claim 1, wherein the effective amount of LIF or a variant thereof, or a biologically active fragment of LIF or a variant thereof administered is between 0.25-16 micrograms/kg or between 16-50 micrograms/kg, and this amount is administered once or twice per day until symptoms of the disease are gone, the subject no longer responds to treatment or the symptoms of the disease have reached an acceptable level.

8. The method of claim 1, wherein the administration of LIF or variant thereof or a biologically active fragment of LIF or variant thereof continues for 1 to 2 days, 2 to 4 days, 4 to 7 days, 2 weeks to 4 weeks or until symptoms are gone, the subject no longer responds to treatment or the symptoms of the disease have reached an acceptable level.

9. The method of claim 8, wherein the effective amount of LIF or a variant thereof, or a biologically active fragment of LIF or a variant thereof administered is between 0.25-16 micrograms/kg or between 16-50 micrograms/kg, and is administered once or twice daily.

10. The method of claim 1, wherein the subject receives radiation treatment for cancer or as preparation for a bone marrow transplant, and the therapeutic amount LIF or variant thereof or a biologically active fragment of LIF or a variant thereof is administered between 1 to 2 days, 2 to 4 days, 7 days, 2 weeks or up to 1 month before the subject receives radiation treatment to reduce or otherwise ameliorate gastrointestinal radiation injury or GvHD.

11. The method of claim 1, wherein the subject receives radiation treatment for cancer or as preparation for a bone marrow transplant, and the therapeutic amount LIF or variant thereof or a biologically active fragment of LIF or a variant thereof is administered to the subject once or twice daily for the duration of radiation treatment.

12. The method of claim 1, wherein the subject receives radiation treatment for cancer or as preparation for a bone marrow transplant, and the therapeutic amount LIF or variant thereof or a biologically active fragment of LIF or a variant thereof is administered once or twice daily for up to 45 days following the radiation treatment.

13. A method for maintaining or increasing intestinal epithelial cell (IEC) growth in isolated mammalian intestine tissue or in artificial intestine comprising mammalian ISC and IEC, comprising contacting the isolated or artificial intestine in vitro with LIF or a variant thereof, a biologically active fragment of LIF or a variant thereof in an amount that maintains or increases IEC growth, wherein the amount administered per day is between 0.5 μg/kg and 100 μg/kg, between about 1 μg/kg and 75 μg/kg, between 0.25 μg/kg and 50 μg/kg, 5 μg/kg and 50 μg/kg, between 20 μg/kg and 40 μg/kg, or the amount is selected from the group consisting of 0.5 μg/kg, 1 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, and 60 μg/kg.

14. The method of claim 13, wherein the intestinal tissue is intended for an autologous or a non-autologous transplant.

15. The method of claim 19, wherein the intestinal tissue is contacted with LIF or a variant thereof, a biologically active fragment of LIF or a variant thereof prior to transplantation in an amount that maintains or increases IEC number.

16. A method for treating a subject exposed to a damaging level of radiation, comprising administering a therapeutically effective amount of LIF or a variant thereof, a biologically active fragment of LIF or a variant thereof one or more times per day for as long as symptoms appear, which amount is between 0.5 μg/kg and 100 μg/kg, between about 1 μg/kg and 75 μg/kg, between 0.25 μg/kg and 50 μg/kg, 5 μg/kg and 50 μg/kg, between 20 μg/kg and 40 μg/kg, or the amount is selected from the group consisting of 0.5 μg/kg, 1 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, and 60 μg/kg per day.

17. A pharmaceutical composition formulated for topical application or for enteric absorption in the small or large intestine, comprising a pharmaceutically acceptable excipient and LIF or a variant thereof, or a biologically active fragment of LIF or a variant thereof in an amount between 0.5 μg/kg and 100 μg/kg, between about 1 μg/kg and 75 μg/kg, between 0.25 μg/kg and 50 μg/kg, 5 μg/kg and 50 μg/kg, between 20 μg/kg and 40 μg/kg, or the amount is selected from the group consisting of 0.5 μg/kg, 1 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, and 60 μg/kg.

18. The composition of claim 17, wherein LIF or a variant thereof, or a biologically active fragment of LIF or a variant thereof is a recombinant human peptide.

19. The composition of claim 18, wherein the recombinant human LIF peptide is identified by SEQ ID NO: 1, and the biologically active fragment of LIF is identified by SEQ ID NO:2, or is an active fragment comprising SEQ ID NO: 2.

20. The composition of claim 17, wherein composition is formulated for absorption by either the small or large intestine.

21. The method of claim 1, wherein the LIF is human LIF is identified by SEQ ID NO: 1 or variants thereof, and the biologically active fragment of LIF is identified by SEQ ID NO:2. or variants thereof, or is an active fragment comprising SEQ ID NO: 2, or variants thereof.

22. A method for treating GvHD following a solid tumor transplant or blood transfusion, comprising administering a therapeutically effective amount of LIF or a variant thereof, a biologically active fragment of LIF or a variant thereof one or more times per day for as long as symptoms appear, which amount is between 0.5 μg/kg and 100 μg/kg, between about 1 μg/kg and 75 μg/kg, between 0.25 μg/kg and 50 μg/kg, 5 μg/kg and 50 μg/kg, between 20 μg/kg and 40 μg/kg, or the amount is selected from the group consisting of 0.5 μg/kg, 1 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, and 60 μg/kg per day.


SEQUENCE LISTING (16 sequences)

SEQ ID NO: 1 - protein, 202 aa, Homo sapiens
  misc_feature: Human LIF canonical sequence, Isoform A
MKVLAAGVVPLLLVLHWKHGAGSPLPITPVNATCAIRHPCHNNLMNQIRSQLAQLNGSANALFILYYTAQGEPFPNNLDKLCGPNVTDFPPFHANGTEKAKLVELYRIVVYLGTSLGNITRDQKILNPSALSLHSKLNATADILRGLLSNVLCRLCSKYHVGHVDVTYGPDTSGKDVFQKKKLGCQLLGKYKQIIAVLAQAF

SEQ ID NO: 2 - protein, 111 aa, Homo sapiens
  misc_feature: Amino acids 50-160 of the isoform 1 (SEQ ID NO 1)
SQLAQLNGSANALFILYYTAQGEPFPNNLDKLCGPNVTDFPPFHANGTEKAKLVELYRIVVYLGTSLGNITRDQKILNPSALSLHSKLNATADILRGLLSNVLCRLCSKYH

SEQ ID NO: 3 - DNA, 18 bp, Artificial Sequence
  Synthetic Olfm4 Forward
gccactttccaatttcac

SEQ ID NO: 4 - DNA, 18 bp, Artificial Sequence
  Synthetic Olfm4 Reverse
gagcctcttctcatacac

SEQ ID NO: 5 - DNA, 20 bp, Artificial Sequence
  Synthetic Lyz Forward
ggtggtgagagatccccaag

SEQ ID NO: 6 - DNA, 20 bp, Artificial Sequence
  Synthetic Lyz Reverse
cagactccgcagttccgaat

SEQ ID NO: 7 - DNA, 19 bp, Artificial Sequence
  Synthetic Axin 2 Forward
tgagatccacggaaacagc

SEQ ID NO: 8 - DNA, 19 bp, Artificial Sequence
  Synthetic Axin 2 Reverse
gtggctggtgcaaagacat

SEQ ID NO: 9 - DNA, 20 bp, Artificial Sequence
  Synthetic Ascl 2 Forward
tccagttggttagggggcta

SEQ ID NO: 10 - DNA, 20 bp, Artificial Sequence
  Synthetic Ascl 2 Reverse
gcataggcccaggtttcttg

SEQ ID NO: 11 - DNA, 22 bp, Artificial Sequence
  Synthetic Lgr5 Forward
cctactcgaagacttacccagt

SEQ ID NO: 12 - DNA, 21 bp, Artificial Sequence
  Synthetic Lgr5 Reverse
gcattggggtgaatgatagca

SEQ ID NO: 13 - DNA, 20 bp, Artificial Sequence
  Synthetic Wnt3 Forward
cttctaatggagccccacct

SEQ ID NO: 14 - DNA, 21 bp, Artificial Sequence
  Synthetic Wnt3 Reverse
gaggccagagatgtgtactgc

SEQ ID NO: 15 - DNA, 30 bp, Artificial Sequence
  Synthetic Beta-Actin Forward
gaaccctaaggccaaccgtgaaaagatgac

SEQ ID NO: 16 - DNA, 23 bp, Artificial Sequence
  Synthetic Beta-Actin Reverse
gcaggatggcgtgagggagagca